(19) United States
(12) Patent Application Publication
(10) Pub. No.: US 2016/0008298 A1
(43) Pub. Date: Jan. 14, 2016

(54) XANTHOHUMOL-BASED COMPOUNDS AND COMPOSITIONS THEREOF, AND METHODS OF MAKING AND USING THE SAME

(71) Applicant: Oregon State University, Corvallis, OR (US)

(72) Inventor: Jan Frederik Stevens, Corvallis, OR (US)

(73) Assignee: Oregon State University

(21) Appl. No.: 14/798,393

(22) Filed: Jul. 13, 2015

Related U.S. Application Data
(60) Provisional application No. 62/024,438, filed on Jul. 14, 2014.

(51) Int. Cl. A61K 31/12; A61K 47/12; A23L 1/30; A61K 31/59; C07C 45/65; A61K 47/26; A61K 47/10

(52) U.S. Cl. CPC A61K 31/12; A61K 47/26; A61K 47/12; A61K 47/10; A61K 31/59; C07C 45/65; A23L 1/3008; A23L 1/30; A23V 2002/00

(57) ABSTRACT

Disclosed herein are compounds capable of ameliorating, treating, and preventing metabolic syndrome, or risk factors associated with metabolic syndrome, such as obesity, diabetes, and/or cardiovascular disease. In some embodiments, the compounds can function as mild mitochondrial uncouplers and thereby reduce markers of metabolic syndrome by improving insulin sensitivity and glucose metabolism. Methods of making the compounds also are disclosed herein, along with compositions and formulations suitable for administration to a subject. In some embodiments, the compounds (or compositions thereof) can be formulated and administered as dietary supplements.

### CROSS REFERENCE TO RELATED APPLICATION

This application claims the benefit of the earlier filing date of U.S. Provisional Patent Application No. 62/024,438, filed on Jul. 14, 2014, the entirety of which is incorporated herein by reference.

### FIELD

The present disclosure concerns compounds, such as xanthohumol and derivatives thereof, for use in treating, ameliorating, or preventing metabolic syndrome. Also disclosed are compositions of such compounds, and methods of making and using the same.

### BACKGROUND

In spite of educational outreach and new medical procedures, cardiovascular disease remains the number one cause of death worldwide. According to the National Heart, Lung, and Blood Institute, cardiovascular diseases were responsible for more than 811,000 deaths in 2008 alone. This also resulted in an estimated medical care cost of 179.3 billion U.S. dollars. Cardiovascular disease has many underlying causes, though by far the most prominent cause in developed countries is obesity.

An individual's risk of cardiovascular disease is strongly correlated to a series of risk factors, collectively referred to as metabolic syndrome. Metabolic syndrome, characterized by a collection of risk factors including abdominal obesity, insulin insensitivity, elevated plasma triglycerides, hypertension and low plasma high-density lipoproteins (HDLs), is a major contributing factor to the development of cardiovascular diseases and type II diabetes (T2D). Any combination of three or more of these conditions greatly multiplies cardiovascular disease risk.

Mitochondrial uncoupling is a process that alters the normal functioning of the electron transport chain and ATP synthase in the mitochondria. An uncoupling agent will allow protons to reenter the mitochondrial matrix and bypass ATP synthase, partially dissipating the electrochemical gradient established by the electron transport chain. Additional reducing equivalents such as NADH and FADH2 are then needed to reestablish the proton gradient, resulting in higher energy expenditure and additional heat generation as the activity of electron transport chain proteins is increased and additional oxygen is reduced to water. Changes in cellular oxidative phosphorylation rates can therefore be estimated by the rate of oxygen consumption over time in a closed system.

Successful use of medicinal treatments to treat metabolic syndrome has remained a significant challenge. Much interest has been generated regarding the various health benefits of xanthohumol (“XN”), including improved cognitive flexibility in mice and its potential as a chemotherapeutic agent. There exists a need in the art for medicinal treatments for metabolic syndrome that utilize compounds that are safe and readily available.

### SUMMARY

Disclosed herein are embodiments of a pharmaceutical composition for use in treating, ameliorating, or preventing metabolic syndrome, obesity, diabetes, and/or cardiovascular disease. In some embodiments, the pharmaceutical composition comprises a mitochondrial uncoupler having a Formula I

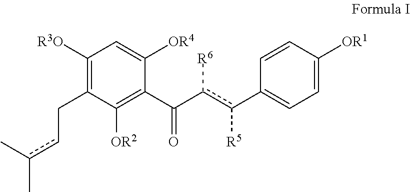

wherein each of R¹, R², R³, and R⁴ independently are selected from hydrogen, aliphatic, heteroaliphatic, haloaliphatic, heteroaryl, aryl, amide, or a prodrug; each of R⁵ and R⁶ independently are selected from halogen, thioether, ascorbic acid, or an ascorbic acid adduct; and at least two pharmaceutically acceptable excipients selected from a fatty acid, an alkylene polyol, and a polysorbate. The pharmaceutical composition can be formulated for administration as a dietary supplement. In some embodiments, each of R¹, R², R³, and R⁴ independently is selected from hydrogen, alkyl, alkenyl, alkynyl, haloalkyl, haloalkenyl, haloalkynyl, aldehyde, ester, phosphate, phosphonate, sulfonate, or sulfonyl. In particular disclosed embodiments, the mitochondrial uncoupler compound can be selected from any one of Formulas II-V disclosed herein. In exemplary embodiments, the mitochondrial uncoupler compound can be selected from:

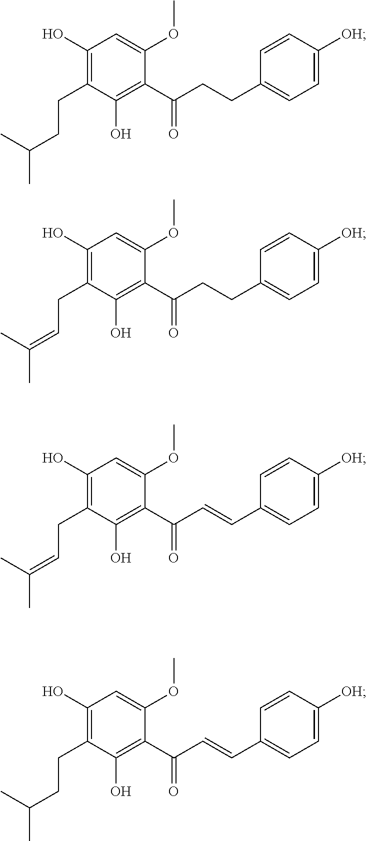 [SMILES: COc1cc(O)c(CCC(C)C)c(O)c1C(=O)CCc1ccc(O)cc1 ; COc1cc(O)c(CC=C(C)C)c(O)c1C(=O)CCc1ccc(O)cc1 ; COc1cc(O)c(CC=C(C)C)c(O)c1C(=O)/C=C/c1ccc(O)cc1 ; COc1cc(O)c(CCC(C)C)c(O)c1C(=O)/C=C/c1ccc(O)cc1]

or a hemi-succinate ester prodrug thereof.

In some embodiments, the at least two pharmaceutically acceptable excipients are selected from oleic acid, ethylene glycol, propylene glycol, polysorbate 80, polysorbate 60, polysorbate 40, polysorbate 20, or combinations thereof. The mitochondrial uncoupler compound can be present in an amount ranging from 0.1 mg to 500 mg per dosage. In yet other embodiments, the mitochondrial uncoupler compound is present in an amount ranging from 45 mg to 180 mg per dosage and the compound is

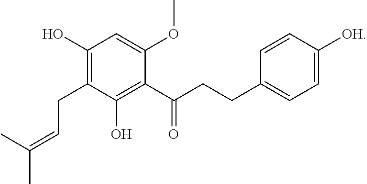 [SMILES: COc1cc(O)c(CC=C(C)C)c(O)c1C(=O)CCc1ccc(O)cc1]

In some embodiments, the pharmaceutical composition can further comprise one or more additional mitochondrial uncoupler compounds having a formula selected from one or more of Formulas I or II-V. In some embodiments, the pharmaceutical composition is used to treat, ameliorate, or prevent metabolic syndrome, obesity, and/or cardiovascular disease wherein the mitochondrial uncoupler compound has a formula

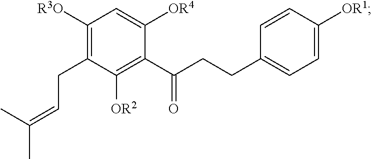

the at least two pharmaceutically acceptable excipients are selected from oleic acid, propylene glycol, and polysorbate 80; and the composition does not comprise a drug for regulating insulin levels or a compound or extract derived from acacia. In such embodiments, the mitochondrial uncoupler compound is

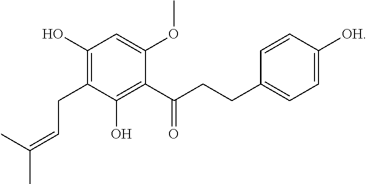 [SMILES: COc1cc(O)c(CC=C(C)C)c(O)c1C(=O)CCc1ccc(O)cc1]

And, in some embodiments, the pharmaceutical composition can further comprise vitamin D.

Also disclosed herein are embodiments of a method of treating, ameliorating, or preventing metabolic syndrome, obesity, diabetes, and/or cardiovascular disease, comprising administering to a subject a pharmaceutical composition formulated to deliver a therapeutically effective amount of a mitochondrial uncoupler compound having a formula

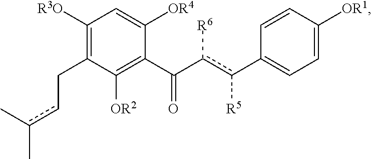

wherein each of R¹, R², R³, and R⁴ independently are selected from hydrogen, aliphatic, heteroaliphatic, haloaliphatic, heteroaryl, aryl, amide, or a prodrug; each of R⁵ and R⁶ independently are selected from halogen, thioether, ascorbic acid, or an ascorbic acid adduct; and wherein the pharmaceutical composition further comprises two or more pharmaceutically acceptable excipients selected from oleic acid, an alkylene polyol, and a polysorbate. In some embodiments, the therapeutically effective amount ranges from 0.1 mg/day to 500 mg/day. In some embodiments, the mitochondrial uncoupler compound has a structure

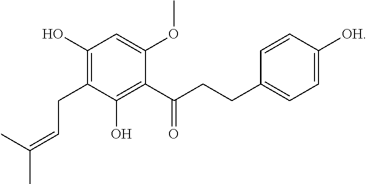 [SMILES: COc1cc(O)c(CC=C(C)C)c(O)c1C(=O)CCc1ccc(O)cc1]

The method can comprise administering to a subject a therapeutically effective amount ranging from 45 mg to 180 mg. In some embodiments, administering the mitochondrial uncoupler compound to the subject provides increased glucose tolerance. In yet additional embodiments, administering the mitochondrial uncoupler compound to the subject prevents weight gain or reduces body weight in the subject.

Also disclosed herein are embodiments of a method of making a mitochondrial uncoupler compound, comprising exposing a transition metal catalyst in an ionic liquid solution to a starting material having a formula

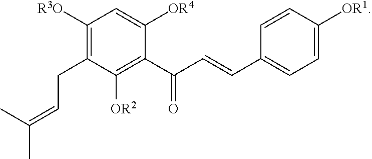

In some embodiments, the method comprises using a transition metal catalyst that comprises a transition metal selected from Rh or Pd. In some embodiments, the transition metal catalyst is RhCl(PPh₃)₃ and the ionic liquid comprises 1-butyl-3-methylimidazolium tetrafluoroborate.

The foregoing and other objects, features, and advantages of the present disclosure will become more apparent from the following detailed description, which proceeds with reference to the accompanying figures.

### BRIEF DESCRIPTION OF THE DRAWINGS

FIG. 1 shows common structures of flavonoids, chalcones and xanthohumol.

FIG. 2 shows the metabolic conversion of xanthohumol (“XN”) into desmethylxanthohumol (“DMX”) and 6- and 8-prenylnaringenin (“6-PN” and “8-PN”); XN is spontaneously converted into isoxanthohumol (“IX”) by intramolecular Michael addition and 8-PN is formed from DMX or from IX by CYP-mediated demethylation.

FIG. 3 shows an exemplary conversion of α,β-dihydroxanthohumol (“DXN”) from XN.

FIG. 4 is a graph illustrating results establishing that DXN causes mitochondrial uncoupling in cells.

FIG. 5 is a graph showing a comparison of XN, DXN and tetrahydroxanthohumol (“TXN”) as mild mitochondrial uncouplers, wherein DXN and TXN cause mitochondrial uncoupling in cells; mouse skeletal C2C12 cells were sequentially treated with oligomycin (1 μM) and 5 μM of the TXN, DXN, and XN, and 2 μM DXN.

FIG. 6 is a bar graph showing a comparison of XN, DXN and TXN as mild mitochondrial uncouplers, with DXN and TXN being more potent than XN in this particular embodiment.

FIG. 7 is a graph showing a comparison of XN, DXN and TXN as mild mitochondrial uncouplers, wherein XN, DXN, and TXN reduce lactate formation following oligomycin treatment as a result of mitochondrial uncoupling in cells; C2C12 cells were sequentially treated with oligomycin (1 μM) and 5 μM of XN, DXN and TXN (see FIG. 5 for legend).

FIG. 8 is a bar graph showing a comparison of XN, DXN and TXN as mild mitochondrial uncouplers using the reduction of the extracellular acidification rate (ΔECAR) as an indirect measurement of the test compounds' ability to increase oxygen consumption.

FIG. 9 is a graph showing a comparison of XN, DXN and TXN as mild mitochondrial uncouplers, wherein DXN and TXN cause mitochondrial uncoupling in cells; C2C12 cells were sequentially treated with oligomycin (1 μM) and 5 μM of the XN, DXN and TXN.

FIG. 10 is a bar graph showing a comparison of XN, DXN and TXN as mild mitochondrial uncouplers.

FIGS. 11A and 11B are bar graphs showing that after one hour of exposure (FIG. 11B), XN, DXN and TXN did not significantly affect cell viability up to 50 μM; and after 24 hours of exposure (FIG. 11A), cell viability was decreased at 8 μM of DXN, 25 μM of TXN and 50 μM of XN; cell viability of drug-treated cells is displayed as a percentage of control cells (i.e., cells not treated) and the * symbol indicates p<0.05.

FIG. 12 is a graph showing DXN causes mitochondrial uncoupling in cells; C2C12 cells were sequentially treated with oligomycin (1 μM) and the indicated concentration of DXN (uncoupler), wherein * indicates p<0.05 (by ANOVA procedure in PROC MIXED).

FIG. 13 is a bar graph establishing that DXN causes mitochondrial uncoupling in cells, wherein changes in OCR and ECAR after injection of test compounds are plotted as bars (mean±SEM, n=3), and * indicates p<0.05 (by ANOVA procedure in PROC MIXED).

FIG. 14 is a graph showing that DXN causes mitochondrial uncoupling in cells; C2C12 cells were sequentially treated with oligomycin (1 μM) and the indicated concentration of DXN (uncoupler), wherein * indicates p<0.05 (by ANOVA procedure in PROC MIXED); see legend in FIG. 12.

FIG. 15 is a bar graph establishing that DXN causes mitochondrial uncoupling in cells, wherein changes in OCR and ECAR after injection of test compounds is plotted as bars (mean±SEM, n=3), and * indicates p<0.05 (by ANOVA procedure in PROC MIXED).

FIG. 16 is a graph establishing that DXN causes mitochondrial uncoupling in cells; C2C12 cells were sequentially treated with oligomycin (1 μM) and the indicated concentration of DXN (uncoupler), wherein * indicates p<0.05 (by ANOVA procedure in PROC MIXED).

FIG. 17 is a bar graph establishing DXN causes mitochondrial uncoupling in cells, wherein changes in OCR and ECAR after injection of test compounds are plotted as bars (mean±SEM, n=3), and * indicates p<0.05 (by ANOVA procedure in PROC MIXED).

FIG. 18 is a bar graph establishing that XN, DXN and TXN are able to depolarize the mitochondrial transmembrane potential measured as a decrease in the ratio aggregate/monomers of the JC-1 dye using the commercially available JC-1 assay kit; * indicates p<0.05.

FIG. 19 shows a hydrogenation mechanism using a Wilkinson catalyst; the steps illustrated in FIG. 19 include (1) addition, (2) alkene addition, (3) migratory insertion, and (4) reductive elimination of the alkane, regeneration of the catalyst.

FIGS. 20A-20F illustrate the structure of DXN (FIG. 20A), as well as exemplary characterization data of DXN (FIGS. 20B-20F).

FIG. 21 shows XN, DXN, TXN chemical structures.

FIG. 22 is a bar graph showing cellular uptake of XN, DXN, and TXN in C2C12 cells; percentages are of total amount of compound applied to cells, and differences were small, and not statistically significant (error bars indicate standard error of the mean (SEM)).

FIG. 23 is a graph showing average mouse weights over 12 weeks for mice treated with TXN, DXN, and XN; while approximate dose was the same (30 mg/kg body weight/day), the TXN mice gained the least weight on average, followed by DXN and XN, compared to the control.

FIG. 24 is a graph showing plasma glucose levels in high-fat fed mice, treated with test compounds for 4 weeks at a dose level of 30 mg/kg body weight/day, over a period of 2 hours following a 2 g/kg glucose intraperitoneal (i.p.) injection; peak glucose levels were achieved at 15 minutes for XN and TXN, and 30 minutes for DXN and the control mice and the highest average increase in plasma glucose was seen in the control group at approximately 600 mg/dL, while the XN, DXN and TXN groups reached peaks of 508.8, 407.2, and 423.0 mg/dL, respectively.

FIG. 25 is a graph showing glucose tolerance test blood glucose levels over a 2 hour period following a 2 g/kg glucose i.p. injection in week 11 of the feeding trial of high-fat fed mice treated with test compounds at 30 mg/kg body weight/day; all mice showed an extended plasma glucose recovery time, as well as larger glucose increases after injection and in contrast to testing at 4 weeks, few mice had recovered to fasting plasma glucose levels within 2 hours.

FIGS. 26A-26D are bar graphs showing fasting plasma glucose (FIG. 26A), insulin (FIG. 26B), triglycerides (FIG. 26C), and cholesterol (FIG. 26D), measured from plasma collected at time of sacrifice following a 6 hour fast; TXN mice showed significantly lower (p<0.05) glucose and insulin levels compared to control and no differences between treatment groups were observed in cholesterol or triglycerides.

FIG. 27 is a graph showing weekly body weights of C57BL/6J mice fed control, low XN (30 mgkg body weight/day) and high XN (60 mg/kg body weight/day) diets for 12 weeks.

FIG. 28 is a graph showing no difference in food intake between treated and untreated mice for the studies from FIG. 27.

### DETAILED DESCRIPTION

### I. Explanation of Terms

The following explanations of terms are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise.

Unless explained otherwise, all technical and scientific terms used herein have the same meaning as commonly understood to one of ordinary skill in the art to which this disclosure belongs. The compounds, methods, and examples are illustrative only and not intended to be limiting, unless otherwise indicated. Other features of the disclosure are apparent from the following detailed description and the claims. Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that can depend on the desired properties sought and/or limits of detection under standard test conditions/methods. When directly and explicitly distinguishing embodiments from discussed prior art, the embodiment numbers are not approximates unless the word “about” is recited.

Furthermore, not all alternatives recited herein are equivalents. To facilitate review of the various embodiments of the disclosure, the following explanations of specific terms are provided. Certain functional group terms include an R^{a} group that, though not part of the defined functional group, indicates how the functional group attaches to the compound to which it is bound.

Aldehyde: R^{a}C(O)H, wherein R^{a} is the atom of the formulas disclosed herein to which the aldehyde group is attached.

Aliphatic: A hydrocarbon, or a radical thereof, having at least one carbon atom to 50 carbon atoms, such as one to 25 carbon atoms, or one to ten carbon atoms, and which includes alkanes (or alkyl), alkenes (or alkenyl), alkynes (or alkynyl), including cyclic versions thereof, and further including straight- and branched-chain arrangements, and all stereo and positional isomers as well.

Alkyl: A saturated monovalent hydrocarbon having at least one carbon atom to 50 carbon atoms, such as one to 25 carbon atoms, or one to ten carbon atoms, wherein the saturated monovalent hydrocarbon can be derived from removing one hydrogen atom from one carbon atom of a parent compound (e.g., alkane). An alkyl group can be branched, straight-chain, or cyclic (e.g., cycloalkyl).

Alkenyl: An unsaturated monovalent hydrocarbon having at least two carbon atoms 50 carbon atoms, such as two to 25 carbon atoms, or two to ten carbon atoms, and at least one carbon-carbon double bond, wherein the unsaturated monovalent hydrocarbon can be derived from removing one hydrogen atom from one carbon atom of a parent alkene. An alkenyl group can be branched, straight-chain, cyclic (e.g., cylcoalkenyl), cis, or trans (e.g., E or Z).

Alkynyl: An unsaturated monovalent hydrocarbon having at least two carbon atoms 50 carbon atoms, such as two to 25 carbon atoms, or two to ten carbon atoms and at least one carbon-carbon triple bond, wherein the unsaturated monovalent hydrocarbon can be derived from removing one hydrogen atom from one carbon atom of a parent alkyne. An alkynyl group can be branched, straight-chain, or cyclic (e.g., cycloalkynyl).

Amide: R^{a}C(O)NR^{b}R^{c} wherein R^{a} is the atom of the formulas disclosed herein to which the amide is attached, and each of R^{b} and R^{c} independently is selected from hydrogen, aliphatic, heteroaliphatic, aryl, heteroaryl, or any combination thereof.

Amine: R^{a}NR^{b}R^{c}, wherein R^{a} is the atom of the formulas disclosed herein to which the amine is attached, and each of R^{b} and R^{c} independently is selected from hydrogen, alkyl, alkenyl, alkynyl, aryl, heteroalkyl, heteroalkenyl, heteroalkynyl, heteroaryl, and any combination thereof.

Aryl: An aromatic carbocyclic group comprising at least five carbon atoms to 15 carbon atoms, such as five to ten carbon atoms, having a single ring or multiple condensed rings, which condensed rings can or may not be aromatic provided that the point of attachment is through an atom of the aromatic carbocyclic group.

Carboxyl: R^{a}C(O)OR^{b}, wherein R^{a} is the atom of the formulas disclosed herein to which the carboxyl group is attached and wherein R^{b} is alkyl, alkenyl, alkynyl, aryl, heteroalkyl, heteroalkenyl, heteroalkynyl, heteroaryl, hydrogen, and any combination thereof.

Derivative: A compound that is derived from a parent compound (e.g., a structurally similar compound) or a compound that can be imagined to arise from another compound, for example, if one atom or functional group is replaced with another atom, group of atoms, or another functional group.

Ester: R^{a}C(O)OR^{b}, wherein R^{a} is the atom of the formulas disclosed herein to which the ester group is attached and R^{b} is selected from hydrogen, alkyl, alkenyl, alkynyl, aryl, heteroalkyl, heteroalkenyl, heteroalkynyl, or heteroaryl.

Haloaliphatic: An aliphatic group wherein one or more hydrogen atoms, such as one to 10 hydrogen atoms, independently is replaced with a halogen atom, such as fluoro, bromo, chloro, or iodo.

Haloalkyl: An alkyl group wherein one or more hydrogen atoms, such as one to 10 hydrogen atoms, independently is replaced with a halogen atom, such as fluoro, bromo, chloro, or iodo. In an independent embodiment, haloalkyl can be a CX₃ group, wherein each X independently can be selected from fluoro, bromo, chloro, or iodo.

Heteroaliphatic: An aliphatic group comprising at least one heteroatom to 20 heteroatoms, such as one to 15 heteroatoms, or one to 5 heteroatoms, which can be selected from, but not limited to oxygen, nitrogen, sulfur, selenium, phosphorous, and oxidized forms thereof within the group.

Heteroalkyl/Heteroalkenyl/Heteroalkynyl: An alkyl, alkenyl, or alkynyl group (which can be branched, straight-chain, or cyclic) comprising at least one heteroatom to 20 heteroatoms, such as one to 15 heteroatoms, or one to 5 heteroatoms, which can be selected from, but not limited to oxygen, nitrogen, sulfur, selenium, phosphorous, and oxidized forms thereof within the group.

Heteroaryl: An aryl group comprising at least one heteroatom to six heteroatoms, such as one to four heteroatoms, which can be selected from, but not limited to oxygen, nitrogen, sulfur, selenium, phosphorous, and oxidized forms thereof within the ring. Such heteroaryl groups can have a single ring or multiple condensed rings, wherein the condensed rings may or may not be aromatic and/or contain a heteroatom, provided that the point of attachment is through an atom of the aromatic heteroaryl group.

Mitochondrial Uncoupler: A compound that can allow protons to reenter a mitochondrial matrix and bypass ATP synthase, partially dissipating the electrochemical gradient established by the electron transport chain.

Prodrug: Prodrugs include compounds disclosed herein that comprise at least one progroup. Prodrugs may be active in their prodrug form, or may be inactive until converted under physiological or other conditions of use to an active drug form. In some embodiments, one or more functional groups of the compounds disclosed herein are used to attach progroups that can be released from the compound under conditions of use, such as through hydrolysis, enzymatic cleavage or some other cleavage mechanism, to yield the functional groups originally present on the compound prior to adding the progroup. Solely by way of example, hydroxyl groups may be reacted with another compound to form an ester, ether, phosphate, phosphonate, sulfonate, or sulfonyl progroup that cleaves under conditions of use to re-generate the hydroxyl group. Functional groups of the compounds disclosed herein that may be masked with progroups for inclusion in a progroup include, but are not limited to, amines (primary and secondary), dicarboxylic acids (e.g., succinic acid), hydroxyls, sulfanyls (thiols), carboxyls, carbonyls, phenols, and the like. In some embodiments, exemplary progroups that cleave to yield hydroxyl groups that can be included in the prodrugs of the present disclosure include, but are not limited to, sulfonates, sulfonyls, ethers, esters, carbonates, phosphates, phosphonates, and the like.

Subject: This term refers to any mammal, such as humans, and non-human mammals (e.g., domestic animals, non-domestic animals, zoo animals, and farm animals).

Sulfonyl: A functional group having a formula R^{a}SO₂R^{b}, wherein R^{a} is the atom of the formulas disclosed herein to which the sulfonyl is attached, and R^{b} is selected from hydrogen, aliphatic, heteroaliphatic, aryl, heteroaryl, and any combination thereof.

Sulfonate: A functional group having a formula R^{a}SO₂OR^{b}, wherein R^{a} is the atom of the formulas disclosed herein to which the sulfonate is attached, and R^{b} is selected from hydrogen, aliphatic, heteroaliphatic, aryl, heteroaryl, and any combination thereof.

A person of ordinary skill in the art would recognize that the definitions provided above are not intended to include impermissible substitution patterns (e.g., methyl substituted with 5 different groups, and the like). Such impermissible substitution patterns are easily recognized by a person of ordinary skill in the art. Any functional group disclosed herein and/or defined above can be substituted or unsubstituted, unless otherwise indicated herein.

The following abbreviations also are provided to facilitate understanding of terms used throughout the application.

“8-PN” means 8-prenylnaringenin

“ADP” means adenosine diphosphate

“ATP” means adenosine triphosphate

“Δψm” means transmembrane (electrical) potential

“DNP” means dinitrophenol

“DXN” means α,β-dihydroxanthohumol

“ETC” means electron transfer chain

“FAD” means flavin adenine dinucleotide, fully oxidized form

“FADH2” means flavin adenine dinucleotide, reduced form

“FBS” means fetal bovine serum

“FCCP” means carbonyl cyanide-4-(trifluoromethoxy)phenylhydrazone

“NAD+” means nicotinamide adenine dinucleotide, oxidized form

“NADH” means nicotinamide adenine dinucleotide, reduced form

“OCR” means oxygen consumption rate

“P/O” means ATP/Oxygen ratio

“ROS” means reactive oxygen species

“TXN” means tetrahydroxanthohumol

“UCP” means uncoupling protein

“XN” means xanthohumol

“[bmim][BF4]” means 1-butyl-3-methylimidazolium tetrafluoroborate

### II. Introduction

There is skepticism in the art related to the practice of using conventional mitochondrial uncouplers as therapeutics due to the possible toxicity of such compounds at higher doses and side effects such as plasma membrane depolarization at even low doses. For example, carbonilcyanide p-triflouromethoxyphenylhydrazone (FCCP) has been found to depolarize the plasma membrane from concentrations above 2.5 μM, which is an undesirable effect for use in treating metabolic disorders. Additionally, the integrity of the mitochondrial function has to be controlled; therefore, uncouplers should not affect ADP phosphorylation and cell membrane and mitochondrial enzymes activity.

It has been determined that xanthohumol (XN) has various health benefits, including improved cognitive flexibility. It also can be used as a cancer chemopreventive agent. XN also has been shown to reduce some markers of metabolic syndrome; however, XN comprises an α,β-unsaturated ketone in its chemical structure and therefore can spontaneously form a stable isomer known as isoxanthohumol (IX). IX can be further metabolized, and through several steps can be transformed into a compound known as 8-prenylnaringenin (8-PN). 8-PN is one of the most potent phytoestrogens currently known, and can interact with both α and β estrogen receptors. Because of this reactivity, XN supplementation could be coupled with upregulation of estrogenic pathways. However, it is known that phytoestrogens have potentially disruptive effects that can be amplified if large quantities of XN were being administered continuously and over a long period of time. Accordingly, there exists a need in the art to develop compounds and methods of making and using such compounds that possess the beneficial effects of the XN without the potentially toxic side effects. Furthermore, XN's natural amounts are too small to have a significant effect through a dietary consumption.

As xanthohumol is a potent pro-estrogenic agent, it is therefore important to inhibit or prevent the formation of the estrogenic metabolite of xanthohumol, i.e., 8-prenylnaringenin (8-PN). The inventor of the present disclosure has determined that reduction of the alkene moiety present in XN prevents the formation of the estrogenic metabolite 8-PN. Additionally, the inventors have discovered new XN derivatives that can be made using low-cost, non-toxic reagents and methods. Such XN derivatives display little or no estrogenic activity and therefore are attractive therapeutic compounds for treating metabolic syndrome. The compounds disclosed herein exhibit mild mitochondrial uncoupling effects to help to limit their potency, increasing their safety and enhancing their potential as supplements. “Mild mitochondrial uncoupling effects,” as used herein, means that the compounds have a self-limiting effect because their activity as protonophores decreases when mitochondria reach a higher state of uncoupling and thus the uncoupling induced by the compounds is unlikely to cause death in contrast to conventional uncouplers. For example, dinitrophenol exhibits a mouse LD50 of 45 mg/kg. In contrast, the LD50 of XN is greater 2000 mg/kg in mice, a dose that exceeds the effective dose level by more than 50 times.

The present disclosure concerns compounds useful for treating metabolic syndrome, or risk factors associated with metabolic syndrome, such as obesity, diabetes, and/or cardiovascular disease. In some embodiments, the compounds can be provided as compositions, such as formulations suitable as dietary supplements and/or suitable for oral administration that can be administered to prevent, ameliorate, or treat metabolic syndrome, or risk factors associated with metabolic syndrome, such as obesity, diabetes, and/or cardiovascular disease, in a subject. In particular disclosed embodiments, the compounds can include any compound satisfying the formulas described below, with representative compounds being xanthohumol, derivatives thereof (e.g., α,β-dihydroxanthohumol, tetrahydroxanthohumol, isoxanthohumol and the like), or prodrugs thereof. Without being limited to a particular theory of operation, it is currently believed that the compounds disclosed herein can act as mild mitochondrial uncouplers for treatment of metabolic syndrome, or risk factors associated with metabolic syndrome, such as obesity, diabetes, and/or cardiovascular disease.

### III. Compounds

Disclosed herein are embodiments of compounds that are useful in treating metabolic syndrome, or risk factors associated with metabolic syndrome, such as obesity, diabetes, and/or cardiovascular disease. The compounds also can act as mild mitochondrial uncouplers. In some embodiments, the compounds can have structures satisfying Formula I.

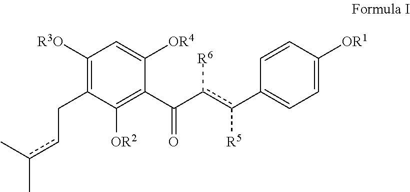

With reference to Formula I, each of R¹, R², R³, and R⁴ independently can be hydrogen, aliphatic, heteroaliphatic, haloaliphatic, heteroaryl, aryl, amide, or a prodrug; and each of R⁵ and R⁶ independently can be selected from halogen, thioether, cycloheteroaliphatic, or combinations thereof. Each of R¹, R², R³, and R⁴ can be the same or different. In particular disclosed embodiments, each of R¹, R² and R³ independently can be selected from hydrogen, alkyl, alkenyl, alkynyl, heteroalkyl, heteroalkenyl, heteroalkynyl, haloalkyl, haloalkenyl, haloalkynyl, aldehyde, ester, sulfonate, sulfonyl, phosphate, or phosphonate. In particular disclosed embodiments, each of R⁵ and R⁶, when present, independently can be selected from Br, Cl, F, I, —SCH₂C(H)(NHC(O)CH₃)C(O)OH, ascorbic acid, or an ascorbic acid adduct. In yet additional embodiments, each of R¹, R², R³, and R⁴ independently can be selected from hydrogen; methyl; ethyl; propyl; butyl; pentyl; —CX₃, —CHX₂, or —CH₂X (wherein each X is selected from Br, Cl, F, or I); —C(O)R^{b}, —C(O)OR^{b}, —C(O)OC(O)OR^{b}, —C(O)NR^{b}R^{c}, —C(O)(CH₂)ₙC(O)OR^{b}, wherein n ranges from 1 to 10, —P(O)(OR^{b})₂, or S(O)₂R^{b}, wherein each of R^{b} and R^{c} independently can be selected from hydrogen, alkyl, alkenyl, alkynyl, aryl, heteroalkyl, heteroalkenyl, heteroalkynyl, or heteroaryl. In some embodiments, R⁵ or R⁶ can be:

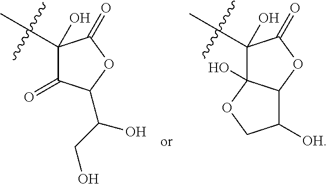 [SMILES: *C1(O)C(=O)OC(C(O)CO)C1=O ; *C1(O)C(=O)OC2C(O)COC21O]

In some embodiments, the compound can be a tautomer of Formula I and therefore can have a structure satisfying Formula IA, below, wherein R⁷ can be any group described above for R¹, R², R³, and R⁴.

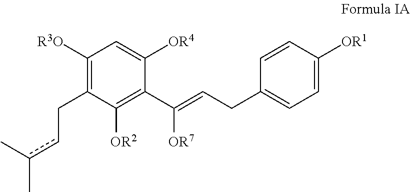

In some embodiments, the compounds can have structures satisfying any one of Formulas II-V.

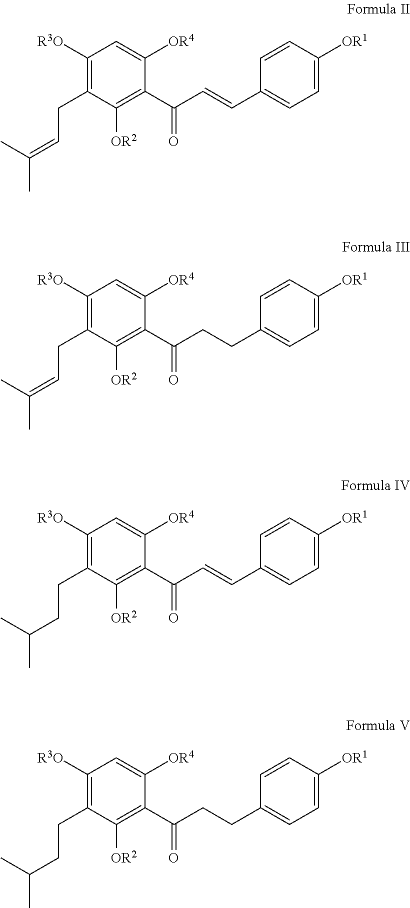

Representative compounds are illustrated in Table 1.

TABLE 1
Representative Compounds

| 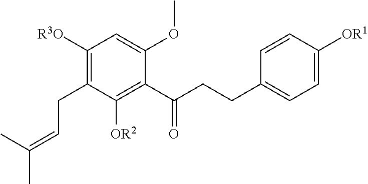 | | | 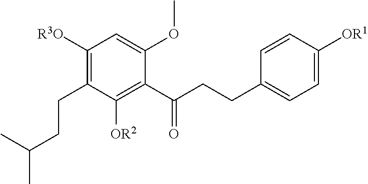 | | |
|---|---|---|---|---|---|
| R¹ | R² | R³ | R¹ | R² | R³ |
| methyl | methyl | methyl | methyl | methyl | methyl |
| ethyl | methyl | methyl | ethyl | methyl | methyl |
| propyl | methyl | methyl | propyl | methyl | methyl |
| butyl | methyl | methyl | butyl | methyl | methyl |
| methyl | ethyl | methyl | methyl | ethyl | methyl |
| methyl | propyl | methyl | methyl | propyl | methyl |
| methyl | butyl | methyl | methyl | butyl | methyl |
| methyl | methyl | ethyl | methyl | methyl | ethyl |
| methyl | methyl | propyl | methyl | methyl | propyl |
| methyl | methyl | butyl | methyl | methyl | butyl |
| methyl | ethyl | ethyl | methyl | ethyl | ethyl |
| ethyl | ethyl | ethyl | ethyl | ethyl | ethyl |
| propyl | ethyl | ethyl | propyl | ethyl | ethyl |
| butyl | ethyl | ethyl | butyl | ethyl | ethyl |
| ethyl | methyl | ethyl | ethyl | methyl | ethyl |
| ethyl | propyl | ethyl | ethyl | propyl | ethyl |
| ethyl | butyl | ethyl | ethyl | butyl | ethyl |
| ethyl | ethyl | methyl | ethyl | ethyl | methyl |
| ethyl | ethyl | propyl | ethyl | ethyl | propyl |
| ethyl | ethyl | butyl | ethyl | ethyl | butyl |
| methyl | propyl | propyl | methyl | propyl | propyl |
| ethyl | propyl | propyl | ethyl | propyl | propyl |
| butyl | propyl | propyl | butyl | propyl | propyl |
| propyl | methyl | propyl | propyl | methyl | propyl |
| propyl | ethyl | propyl | propyl | ethyl | propyl |
| propyl | propyl | propyl | propyl | propyl | propyl |
| propyl | butyl | propyl | propyl | butyl | propyl |
| propyl | propyl | methyl | propyl | propyl | methyl |
| propyl | propyl | ethyl | propyl | propyl | ethyl |
| propyl | propyl | butyl | propyl | propyl | butyl |
| methyl | butyl | butyl | methyl | butyl | butyl |
| ethyl | butyl | butyl | ethyl | butyl | butyl |
| propyl | butyl | butyl | propyl | butyl | butyl |
| butyl | butyl | butyl | butyl | butyl | butyl |
| butyl | methyl | butyl | butyl | methyl | butyl |
| butyl | ethyl | butyl | butyl | ethyl | butyl |
| butyl | propyl | butyl | butyl | propyl | butyl |
| butyl | butyl | methyl | butyl | butyl | methyl |
| butyl | butyl | ethyl | butyl | butyl | ethyl |
| butyl | butyl | propyl | butyl | butyl | propyl |
| C(O)(CH₂)₂C(O)OH | methyl | methyl | C(O)(CH₂)₂C(O)OH | methyl | methyl |
| methyl | C(O)(CH₂)₂C(O)OH | methyl | methyl | C(O)(CH₂)₂C(O)OH | methyl |
| methyl | methyl | C(O)(CH₂)₂C(O)OH | methyl | methyl | C(O)(CH₂)₂C(O)OH |
| C(O)(CH₂)₂C(O)OH | ethyl | ethyl | C(O)(CH₂)₂C(O)OH | ethyl | ethyl |
| ethyl | C(O)(CH₂)₂C(O)OH | ethyl | ethyl | C(O)(CH₂)₂C(O)OH | ethyl |
| ethyl | ethyl | C(O)(CH₂)₂C(O)OH | ethyl | ethyl | C(O)(CH₂)₂C(O)OH |
| C(O)(CH₂)₂C(O)OH | propyl | propyl | C(O)(CH₂)₂C(O)OH | propyl | propyl |
| propyl | C(O)(CH₂)₂C(O)OH | propyl | propyl | C(O)(CH₂)₂C(O)OH | propyl |
| propyl | propyl | C(O)(CH₂)₂C(O)OH | propyl | propyl | C(O)(CH₂)₂C(O)OH |
| C(O)(CH₂)₂C(O)OH | butyl | butyl | C(O)(CH₂)₂C(O)OH | butyl | butyl |
| butyl | C(O)(CH₂)₂C(O)OH | butyl | butyl | C(O)(CH₂)₂C(O)OH | butyl |
| butyl | butyl | C(O)(CH₂)₂C(O)OH | butyl | butyl | C(O)(CH₂)₂C(O)OH |
| C(O)OH | methyl | methyl | C(O)OH | methyl | methyl |
| methyl | C(O)OH | methyl | methyl | C(O)OH | methyl |
| methyl | methyl | C(O)OH | methyl | methyl | C(O)OH |
| C(O)OH | ethyl | ethyl | C(O)OH | ethyl | ethyl |
| ethyl | C(O)OH | ethyl | ethyl | C(O)OH | ethyl |
| ethyl | ethyl | C(O)OH | ethyl | ethyl | C(O)OH |
| C(O)OH | propyl | propyl | C(O)OH | propyl | propyl |
| propyl | C(O)OH | propyl | propyl | C(O)OH | propyl |
| propyl | propyl | C(O)OH | propyl | propyl | C(O)OH |
| C(O)OH | butyl | butyl | C(O)OH | butyl | butyl |
| butyl | C(O)OH | butyl | butyl | C(O)OH | butyl |
| butyl | butyl | C(O)OH | butyl | butyl | C(O)OH |
| C(O)OC(CO)OH | methyl | methyl | C(O)OC(CO)OH | methyl | methyl |
| methyl | C(O)OC(CO)OH | methyl | methyl | C(O)OC(CO)OH | methyl |
| methyl | methyl | C(O)OC(CO)OH | methyl | methyl | C(O)OC(CO)OH |
| C(O)OC(CO)OH | ethyl | ethyl | C(O)OC(CO)OH | ethyl | ethyl |
| ethyl | C(O)OC(CO)OH | ethyl | ethyl | C(O)OC(CO)OH | ethyl |
| ethyl | ethyl | C(O)OC(CO)OH | ethyl | ethyl | C(O)OC(CO)OH |
| C(O)OC(CO)OH | propyl | propyl | C(O)OC(CO)OH | propyl | propyl |
| propyl | C(O)OC(CO)OH | propyl | propyl | C(O)OC(CO)OH | propyl |
| propyl | propyl | C(O)OC(CO)OH | propyl | propyl | C(O)OC(CO)OH |
| C(O)OC(CO)OH | butyl | butyl | C(O)OC(CO)OH | butyl | butyl |
| butyl | C(O)OC(CO)OH | butyl | butyl | C(O)OC(CO)OH | butyl |
| butyl | butyl | C(O)OC(CO)OH | butyl | butyl | C(O)OC(CO)OH |
| H | H | H | H | H | H |
| methyl | H | H | methyl | H | H |
| ethyl | H | H | ethyl | H | H |
| propyl | H | H | propyl | H | H |
| butyl | H | H | butyl | H | H |
| H | methyl | H | H | methyl | H |
| H | ethyl | H | H | ethyl | H |
| H | propyl | H | H | propyl | H |
| H | butyl | H | H | butyl | H |
| H | H | methyl | H | H | methyl |
| H | H | ethyl | H | H | ethyl |
| H | H | propyl | H | H | propyl |
| H | H | butyl | H | H | butyl |
| P(O)(OH)₂ | methyl | methyl | P(O)(OH)₂ | methyl | methyl |
| methyl | P(O)(OH)₂ | methyl | methyl | P(O)(OH)₂ | methyl |
| methyl | methyl | P(O)(OH)₂ | methyl | methyl | P(O)(OH)₂ |
| P(O)(OH)₂ | ethyl | ethyl | P(O)(OH)₂ | ethyl | ethyl |
| ethyl | P(O)(OH)₂ | ethyl | ethyl | P(O)(OH)₂ | ethyl |
| ethyl | ethyl | P(O)(OH)₂ | ethyl | ethyl | P(O)(OH)₂ |
| P(O)(OH)₂ | butyl | butyl | P(O)(OH)₂ | butyl | butyl |
| butyl | P(O)(OH)₂ | butyl | butyl | P(O)(OH)₂ | butyl |
| butyl | butyl | P(O)(OH)₂ | butyl | butyl | P(O)(OH)₂ |

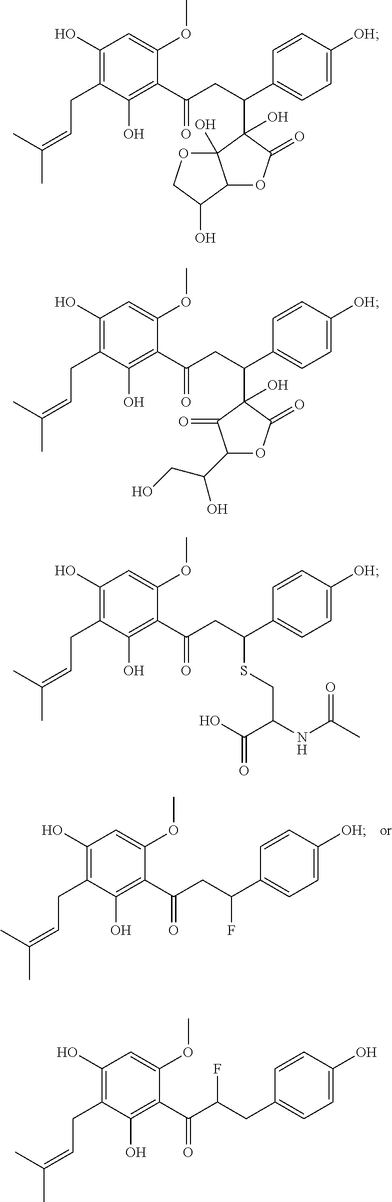

In particular disclosed embodiments, the mitochondrial uncoupler compound is selected

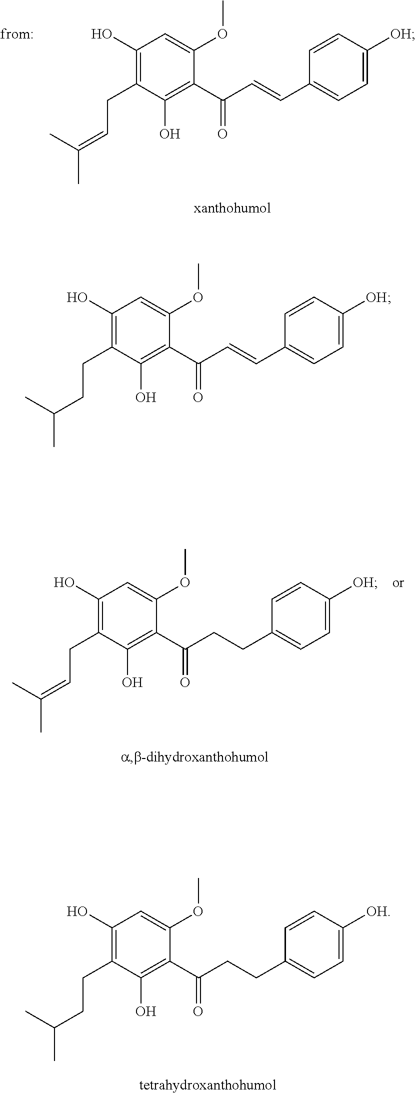 [SMILES: COc1cc(O)c(CC=C(C)C)c(O)c1C(=O)/C=C/c1ccc(O)cc1 ; COc1cc(O)c(CCC(C)C)c(O)c1C(=O)/C=C/c1ccc(O)cc1 ; COc1cc(O)c(CC=C(C)C)c(O)c1C(=O)CCc1ccc(O)cc1 ; COc1cc(O)c(CCC(C)C)c(O)c1C(=O)CCc1ccc(O)cc1]

### IV. Compositions and Formulations

Also disclosed herein are embodiments of compositions (e.g., pharmaceutical compositions) useful for treating, preventing, or ameliorating metabolic syndrome, or risk factors associated with metabolic syndrome, such as obesity, diabetes, and/or cardiovascular disease. In particular disclosed embodiments, the compositions are pharmaceutical compositions formulated for administration as a dietary supplement. The compositions can comprise one or more compounds satisfying the formulas disclosed above. In particular disclosed embodiments, the compositions can comprise one or more mitochondrial uncoupler compounds satisfying any of the formulas disclosed above. In some embodiments, the compositions can further comprise one or more pharmaceutically acceptable excipients, vitamins (e.g., vitamin C, vitamin D, or the like), herbal or botanical products or their extracts (e.g., turmeric, curcumin, resveratrol, grape seed extract, or the like), amino acids, metabolites (e.g., XN—O-glucuronides), extracts (e.g., hop extracts, Ashitaba (Angelica keiskei) extracts, licorice, bitter melon (Mormordica charantia) extracts, or the like), other ingredients, or any combinations thereof.

Exemplary pharmaceutically acceptable excipients can include, but are not limited to, binding agents (e.g., starch, polyvinylpyrrolidone, hydroxypropyl methylcellulose, and the like), fillers (e.g., lactose, cellulose, calcium hydrogen phosphate, and the like), lubricants (e.g., talc, silica, stearates, and the like), emulsifiers/solubilizers (e.g., lecithin; polysorbates; alkylene polyols, such as propylene glycol or ethylene glycol; or fatty acids, such as long-chain fatty acids with aliphatic chains of at least 13 carbon atoms, medium-chain fatty acids with aliphatic chains of between 6 and 12 carbon atoms, and short-chain fatty acids with aliphatic chains of between 2 and 5 carbon atoms), or combinations thereof. Exemplary polysorbates include, but are not limited to, polysorbate 80, polysorbate 60, polysorbate 40, polysorbate 20, or combinations thereof. Exemplary fatty acids include, but are not limited to myristoleic acid, palmitoleic acid, sapienic acid, oleic acid, elaidic acid, vaccenic acid, linoleic acid, arachidonic acid, eicosapentaenoic acid, or combinations thereof. In particular disclosed embodiments, the pharmaceutically acceptable excipient can be selected from oleic acid, propylene glycol, polysorbate 80, and combinations thereof.

In particular disclosed embodiments, the composition may be formulated as a dietary supplement. The term “dietary supplement” in this context concerns a composition comprising an ingestible compound (e.g., a compound satisfying any of the formulas described herein) or composition thereof that provides nutrients (e.g., amino acids, phytochemicals) otherwise not consumed by a subject in sufficient quantity for good health, for protective benefits, or to maintain a normal, healthy lifestyle (such as by preventing disease) as compared to a subject who does not receive the dietary supplement. In exemplary embodiments, the compositions and dietary supplements disclosed herein can comprise one or more of xanthohumol, α,β-dihydroxanthohumol, tetrahydroxanthohumol, isoxanthohumol, or derivatives thereof (e.g., prodrugs). In some embodiments, the dietary supplement can further comprise one or more of the pharmaceutically acceptable excipients disclosed herein. In exemplary embodiments, the composition comprises α,β-dihydroxanthohumol and at least one pharmaceutically acceptable excipient (or at least two pharmaceutically acceptable excipients or at least three pharmaceutically acceptable excipients). In other exemplary embodiments, the composition is a pharmaceutical composition formulated as a dietary supplement comprising α,β-dihydroxanthohumol, oleic acid, polysorbate 80, and propylene glycol.

The compounds (or compositions thereof) can be formulated as a dietary supplement for administration using suitable administration routes, such as oral, nasal, injection, or in a form suitable for administration by inhalation or insufflation.

In particular disclosed embodiments, the compounds (or compositions thereof) are formulated for oral administration. In some embodiments, the compounds (or compositions thereof) are formulated for oral administration by combining at least one compound disclosed herein with one or more pharmaceutically acceptable excipients to form a pharmaceutical composition and forming capsules, lozenges, or tablets that are made of, or contain, the pharmaceutical composition. In yet other embodiments, the compound(s) need not be combined with a pharmaceutically acceptable excipient and can instead be used neat. The capsules, lozenges, or tablets can be coated with films, enteric coatings, sugars, or the like.

In some embodiments, the compounds (and compositions thereof) can be formulated for oral administration by combining at least one compound disclosed herein with one or more pharmaceutically acceptable excipients and an aqueous or non-aqueous delivery medium. Such formulations can be liquid preparations that can take the form of elixirs, solutions, syrups, suspensions, or the like. In some embodiments, the compound (or composition thereof) can be provided as a dry component that can be mixed with an aqueous or non-aqueous delivery medium during administration. Such liquid formulations can further comprise buffer salts, preservatives, flavoring, or coloring.

In some embodiments, the compound (or composition thereof) can be formulated for delayed or controlled release of the compound. For example, the compound can be a prodrug that comprises a progroup that is released (e.g., by metabolic cleavage, hydrolytic cleavage, enzymatic cleavage, or the like) from the compound after administration. In such embodiments, the compound is not pharmacologically active when the progroup is associated with the compound, but becomes pharmacologically active after the progroup is released from the compound.

In particular disclosed embodiments, the compounds (or compositions thereof) are formulated for oral administration so that the composition comprises a therapeutically acceptable amount of the compound, such as an amount ranging from greater than 0 mg to 1000 mg of the compound, such as 5 mg to 500 mg, or 10 mg to 250 mg, or 20 mg to 200 mg, or 50 mg to 150 mg. In particular disclosed embodiments, the compositions comprise a therapeutically effective amount of the compound ranging from 60 mg to 240 mg, or from 45 mg to 180 mg, or from 20 mg to 80 mg, or from 80 mg to 320 mg. In yet additional embodiments, the compositions can be formulated to deliver 0.1 mg/day to 300 mg/day of XN, such as 20 mg/day to 280 mg/day XN, or 60 mg/day to 240 mg/day XN to the subject to which it is administered. In yet additional embodiments, the compositions can be formulated to deliver 0.1 mg to 200 mg of DXN, such as 20 mg to 190 mg of DXN, or 45 mg to 180 mg of DXN to the subject to which it is administered. In yet additional embodiments, the compositions can be formulated to deliver 0.1 mg to 100 mg TXN, such as 10 mg to 90 mg of TXN, or 20 mg to 80 mg of TXN to the subject to which it is administered. In yet additional embodiments, the compositions can be formulated to deliver 0.1 mg to 350 mg of IX, such as 20 mg to 340 mg of IX, or 80 mg to 320 mg of IX to the subject to which it is administered. In yet other embodiments, the formulations can comprise any combination of XN, DXN, TXN, or IX in any of the amount provided above.

The compositions also can comprise pharmaceutically acceptable excipients in a 9:10:10 proportion by weight. In some embodiments, the pharmaceutically acceptable excipients are selected from a fatty acid, an alkylene glycol, and a polysorbate. In some embodiments, the amount of the pharmaceutically excipient can range from greater than 0% to 95%, such as 5% by weight to 90% by weight, or 10% by weight to 80% by weight, or 25% by weight to 45% by weight, or 30% by weight to 35% by weight. In some embodiments, the total volume of the pharmaceutical composition can range from greater than 0 mL to 35 mL per dosage form, such as 0.1 mL to 30 mL per dosage form, or 0.1 mL to 28 mL per dosage form, or 0.6 mL to 1 mL. In exemplary embodiments wherein the pharmaceutical composition is administered via a capsule to a human subject, the volume of the composition that is administered may range from 0.13 mL to 1.3 mL. In some embodiments wherein the composition is formulated for veterinary use, the composition can be administered in amounts ranging from 1.3 mL to 30 mL, such as 1.3 mL to 28 mL. In some embodiments, the animal doses can be derived from the doses described herein by using the FDA formula for allometric interspecies scaling of dose. Such formulas are described in “Guidance for Industry Estimating the Maximum Safe Starting Dose in Initial Clinical Trials for Therapeutics in Adult Healthy Volunteers,” U.S. Department of Health and Human Services, Food and Drug Administration, and Center for Drug Evaluation and Research, July 2005, http://www.fda.gov/cder/guidance/index.htm, which is incorporated herein by reference.

Exemplary formulations are provided below in Table 2. In some embodiments, these exemplary formulations are for human subjects.

TABLE 2
Exemplary dosage and formulation for dietary supplements.

| | Dosage | | | |
|---|---|---|---|---|
| | of active | Oleic | Propylene | Polysorbate |
| Active Ingredient | ingredient | acid | glycol | 80 |
| XN | 0.1-500 mg | 193 mg | 215 mg | 215 mg |
| DXN | 0.1-500 mg | 193 mg | 215 mg | 215 mg |
| TXN | 0.1-500 mg | 193 mg | 215 mg | 215 mg |
| IX | 0.1-500 mg | 193 mg | 215 mg | 215 mg |
| Combinations of XN, | 0.1-500 mg | 193 mg | 215 mg | 215 mg |
| DXN, TXN, and IX | | | | |
| Hop extracts | 0.1-500 mg | 193 mg | 215 mg | 215 mg |
| containing XN at the | | | | |
| specified dosage | | | | |
| Hydrogenated hop | 0.1-500 mg | 193 mg | 215 mg | 215 mg |
| extracts containing | | | | |
| DXN at the specified | | | | |
| dosage | | | | |
| Hydrogenated hop | 0.1-500 mg | 193 mg | 215 mg | 215 mg |
| extracts containing | | | | |
| TXN at the specified | | | | |
| dosage | | | | |
| Hydrogenated hop | 0.1-500 mg | 193 mg | 215 mg | 215 mg |
| extracts containing a | | | | |
| mixture of DXN and | | | | |
| TXN at the specified | | | | |
| total dosage | | | | |

In particular disclosed embodiments, the human equivalent dose of 180 mg XN/day reduced body weight gain by 14% and reduced fasting plasma glucose by 25% when administered to rats. In some embodiments, the human equivalent dose of 60 or 180 mg XN/day reduced the formation of products of dysfunctional fatty acid oxidation by up to 55% when administered to rats. Solely by way of example, the range 0.1-500 mg described above in Table 2 for XN covers the effective human equivalent doses of 60 mg/day to 180 mg/day of XN.

In an independent embodiment, the compositions disclosed herein do not comprise, or are free of a drug for regulating insulin levels, such as biguanides, sulfonylureas, nonsulfonylureas, α-glucosidase inhibitors, and thiazolidinediones. In some independent embodiments, the composition does not comprise or is free of metformin, glipizide, glyburide, glimepiride, rosiglitazone, troglitazone and pioglitazone. In yet another independent embodiment, the compositions disclosed herein do not comprise, or are free of, a compound or extract derived from acacia. In such embodiments, the compound or extract derived from acacia is selected from Acacia catechu or Acacia nilotica (e.g., gum resin, bark powder, heartwood powder, and an Acacia catechu or Acacia nilotica extract).

### V. Methods of Making the Compounds

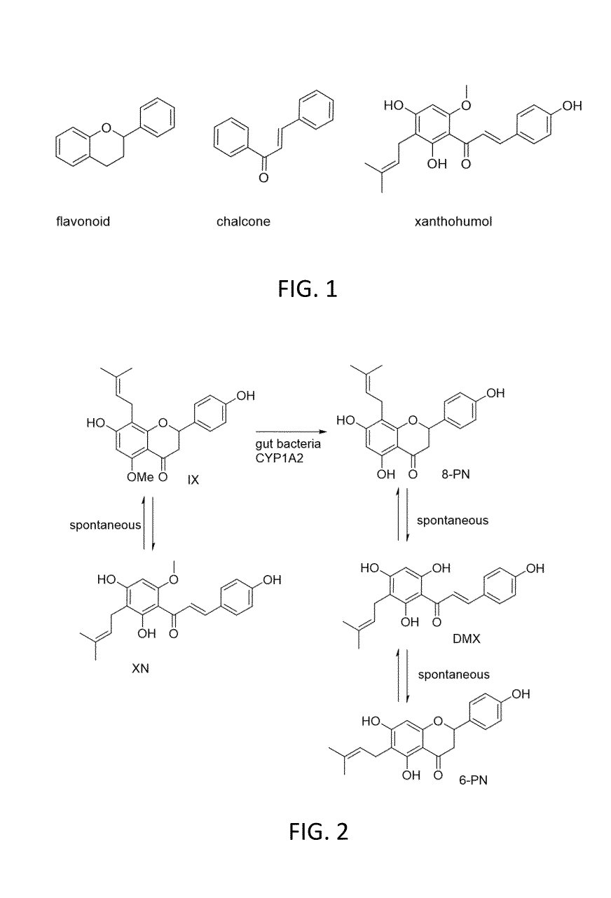

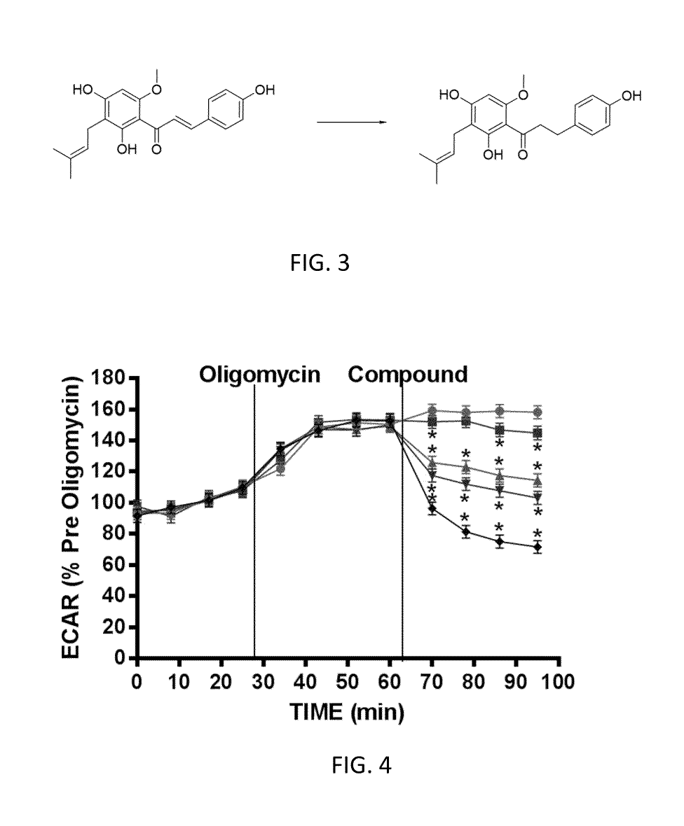

Also disclosed herein are embodiments of methods of making the compounds disclosed herein. In some embodiments, the compounds can be made by derivatizing a parent compound, such as XN or other prenylated chalcone/flavonoid compounds (FIG. 1). The parent compound, such as XN, can be obtained from natural sources or can be chemically synthesized using chemical reagents and starting materials.

In some embodiments, a parent compound can be chemically converted to a derivative compound that can be used in the compositions, formulations, and methods described herein. Some chemical conversions that can be used to make derivatives include, but are not limited to, hydrogenation, etherifcation, esterification, phosphorylation, sulfonylation, substitution, or combinations thereof.

In some embodiments, the hydrogenation of both double bonds of XN produces tetrahydroxanthohumol (TXN). In yet other embodiments, DXN can be produced by selective reduction of the double bond of the α-β unsaturated carbonyl in the parent XN compound. In yet additional embodiments, reduction can be used to make compounds wherein the carbonyl moiety is reduced (such as in a 1,2-reduction), or compounds wherein the XN product is over-reduced to the saturated alcohol derivative.

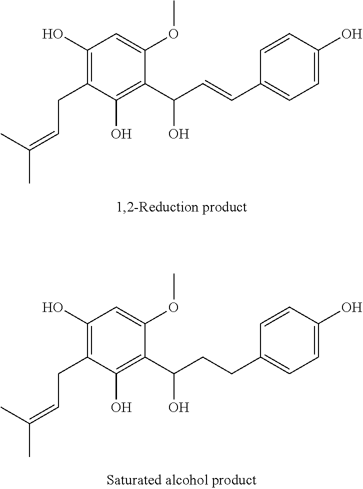 [SMILES: COc1cc(O)c(CC=C(C)C)c(O)c1C(O)/C=C/c1ccc(O)cc1 ; COc1cc(O)c(CC=C(C)C)c(O)c1C(O)CCc1ccc(O)cc1]

In embodiments wherein hydrogenation is used to prepare derivative compounds of XN, catalytic transfer hydrogenation can be used. Such methods avoid the need for expensive equipment and molecular hydrogen gas. In some embodiments, selenium, indium, nickel, palladium catalysts can be used. In yet other embodiments, hydride-based reductions can be used; however, the variation of the chemoselectivity of the reduction depends on the type of hydride agent, the solvent, the substrate and the specific conditions used.

In some embodiments, a palladium-catalyzed reduction system can be used in combination with a reducing agent (e.g., sodium borohydride), a hydrogen donor (e.g., acetic acid), and a solvent (e.g., toluene and dichloromethane). Without being limited to a single theory of operation, it is currently believed that the selectivity of the reaction depends on the solvent used. In some embodiments, non-polar solvents, such as toluene, provide the 1,4-conjugate reduction of the olefinic bond. In some embodiments, polar solvents, such as dichloromethane, can be used to enhance solubility of the parent compound.

In yet other embodiments, rhodium-catalyzed hydrogenation (e.g., using Wilkinson catalyst (RhCl(PPh₃)₃)) selectively provides 1,4-conjugate reduction of the double bond product rather than the 1,2-reduction of the carbonyl product (see mechanism of the catalyzed hydrogenation in FIG. 19). In some embodiments, ionic liquids/solvents can be used as they exhibit good solvent power for many organic chemical reactions, a high miscibility, a high thermal stability, low vapor pressure and other interesting physical properties. An exemplary ionic liquid is [bmim][BF4].

In some embodiments, the derivatives can be obtained by functionalizing particular moieties of the parent compound. Solely by way of example, hydroxyl groups can be converted to haloaliphatic groups, ethers, esters, phosphates, sulfonates, sulfonyls, phosphonates, or any other suitable functional groups described herein using base-catalyzed substitution reactions.

### VI. Methods of Using the Compounds

Also disclosed herein are methods of using the compound, compositions, and formulations disclosed herein as dietary supplements to promote health. In some embodiments, the compounds, compositions, and formulations can be used as dietary supplements that alleviate, treat, or prevent metabolic syndrome, or risk factors associated with metabolic syndrome, such as obesity, diabetes, and/or cardiovascular disease. Without being limited to a single theory of operation, it is currently believed that the compounds disclosed herein can be used as mitochondrial uncouplers.

Mitochondrial uncouplers include compounds that are able to dissipate the proton gradient: they carry protons into the matrix bypassing the proton channel of ATP synthase and therefore they prevent the production of ATP, resulting in a decrease in the coupling efficiency and in the value of the P/O ratio. Such uncoupling agents uncouple the ETC from ADP phosphorylation since they still permit electron transfer along the respiratory chain to O2 to occur even in the absence of ATP synthesis. The oxygen consumption rate is also increased. The “proton leak” across the membrane created by uncouplers decreases the proton motive force and generates heat instead of ATP.

Dinitrophenol (DNP) was efficient at causing weight loss in humans by decreasing coupling efficiency and increasing energy expenditure. DNP has, however, a narrow therapeutic window (potential overdose risk) and dangerous side effects that make this compound not safe as a therapeutic. It is thus still challenging to find an efficient and safe mild mitochondrial uncoupler in order to treat obesity and obesity-related disorders. The compounds disclosed herein are safe, non-toxic, and readily produced and therefore constitute an advancement over the art as a therapeutic for treating metabolic syndrome, or risk factors associated with metabolic syndrome, such as obesity, diabetes, and/or cardiovascular disease, and promoting weight loss.

In particular disclosed embodiments, the method embodiments disclosed herein comprise administering to a subject a therapeutically effective amount of a compound having a structure satisfying any of the formula described herein. In some embodiments, the methods can comprise administering to a subject a pharmaceutical composition comprising a therapeutically effective amount of a mitochondrial uncoupler compound disclosed herein, and one or more pharmaceutically acceptable excipients (e.g., two or more or three or more). In some embodiments, the subject administered the composition can be human or non-human. For example, the composition can be formulated for veterinary use for administration to non-human mammals, such as domestic animals (e.g., dog, cat, horse, rabbit, and the like). In some embodiments, the composition can be formulated as a dietary supplement for use in domestic, farm, zoo, or other animals under the care of humans with the intention to treat, ameliorate, or prevent obesity, cardiovascular disease, and/or diabetes in the animal.

In particular disclosed embodiments, the mitochondrial uncoupler compound is the primary active agent, or even the sole active agent, in the composition that acts to treat, ameliorate, or prevent metabolic syndrome, or risk factors associated with metabolic syndrome, such as obesity, diabetes, and/or cardiovascular disease. Thus, in an independent embodiment, the method does not comprise administering a drug for regulating insulin levels. In yet another independent embodiment, the method does not comprise administering a pharmaceutical composition comprising a drug for regulating insulin levels. In particular disclosed embodiments, the method consists of administering a therapeutically effective amount of a compound having a structure satisfying any of the formulas described herein, or a pharmaceutical composition thereof. In yet other disclosed embodiments, the method consists essentially of administering a therapeutically effective amount of a compound having a structure satisfying any of the formulas described herein, or a pharmaceutical composition thereof. In such methods, any component that deleteriously affects the ability of the compound to effectively treat, ameliorate, or prevent metabolic syndrome, or risk factors associated with metabolic syndrome, such as obesity, diabetes, and/or cardiovascular disease, is omitted, or, in some embodiments of such methods, another pharmaceutically active compound that regulates insulin levels is omitted. In exemplary embodiments of the method, α,β-dihydroxanthohumol is administered to a subject as a dietary supplement alone or in combination with one or more pharmaceutically acceptable excipients.

### VII. Examples

Chemicals:

Xanthohumol, isolated from hops, and tetrahydroxanthohumol were gifts from Dr. Martin Biendl and Dr. Robert Smith, respectively (both from Hopsteiner, Inc., New York). α,β-Dihydroxanthohumol was synthesized according to methods described herein. Oligomycin and the synthetic uncoupler, FCCP (carbonyl cyanide-4-(trifluoromethoxy)phenylhydrazone), were purchased from Seahorse Bioscience (North America, USA). MTT was obtained from Sigma (Missouri, USA). JC-1 staining agent and Cell-Based Assay Buffer tablets were purchased as an assay kit from the company Cayman Chemical (Ann Arbor, USA). The ionic liquid and Wilkinson catalysts were purchased from TCI America, Pd catalyst from Alfa aesar, the sodium borohydride from JT Baker, the ammonium formate from Sigma.

Cell Culture and Treatments:

C2C12 skeletal muscle cells were used because of their high bioenergetics activity and determining the effects of uncouplers on OCR in C2C12 muscle cells was a model of cellular energy generation and expenditure.

Mouse C2C12 skeletal muscle myoblasts (purchased from ATCC, Manassas, Va., USA) were first seeded in 75 cm2 flasks using a culture medium consisting of DMEM (Life Technologies, Grand Island, N.Y., USA), 10% fetal bovine serum (FBS; Atlanta Biologicals, Lawrenceville, Ga., USA), high glucose, L-glutamine, phenol red, 1 mM sodium pyruvate and 100 units/mL penicillin, 100 μg/mL streptomycin. The C2C12 cells were incubated at 37° C. in a humidified atmosphere of 5% CO₂ until they were confluent. Then, they were obtained by trypsinization and seeded at a specific density in the plates of choice depending on the experiment.

MTT Assay:

For cell viability experiments (MTT assay), C2C12 cells were plated in 96-well plates at a density of 4,000 cells per well in 200 μL of growth medium (identical to the culture medium). After 48 hours of incubation, the medium was removed and the cells were treated with fresh solutions of various concentrations of XN, DXN or TXN in phenol red-free growth medium. The final concentrations of XN, DXN, TXN were 1, 2, 5, 8, 10, 25, 50 μM, all in quadruplicate wells, in the 96-well plates. The medium used consists of phenol red free DMEM (Life Technologies, Grand Island, N.Y., USA), 10% FBS, 1% glutamine, 1 mM of sodium pyruvate and 100 units/mL penicillin, 100 μg/mL streptomycin. The plates were then incubated for 24 hours or 1 hour. After removing the medium, the cells were treated with 0.5 mg/mL solution of MTT 3-(4,5-dimethylthiazol-2-yl)-2,5-diphenyltetrazolium bromide in phenol red-free growth medium and incubated for two hours. After removing the medium and adding a solution of acidified isopropanol to each well, the absorbance of each well was measured at 570 nm by using a spectrometer (SpectraMax 190). Cell viability of drug-treated cells is displayed as a percentage of control cells, i.e., cells not treated.

Oxygen Consumption Rate (OCR) and Extracellular Acidification Rate (ECAR) Measurements:

OCR and ECAR measurements were performed with a Seahorse XF 24 Analyzer (Seahorse Bioscience). Cells were plated 24 hours prior to measurements at a density of 40,000 cells per well within growth medium in a 24-well plate. The cells were then allowed to adhere for 24 hours (37° C., 5% CO2). Prior to the assay, the cells were washed with freshly prepared running media consisting of Seahorse XF Assay medium (Seahorse Bioscience, North America, USA), 10 mM glucose and 1 mM sodium pyruvate. The pH of the medium was adjusted to 7.4 with 1 M NaOH. The cells were then equilibrated for one hour at 37° C. without CO₂. The compounds were injected during the assay; OCR and ECAR were measured at 9 minutes measurement intervals. The solutions of oligomycin, FCCP, XN, DXN and TXN were freshly prepared in running medium. The final concentrations in the wells were: 1 μM for oligomycin, 5 μM for XN, DXN and TXN and 2, 5, 8, 25 μM for the dose response experiments with DXN. Ethanol was added to the control wells not to exceed a final concentration of 0.1%. All the compounds were tested in quadruplicate on the 24-well plate. XN is highly non-polar and FBS can be used to dissolve XN. Thus, XN, DXN and TXN were first dissolved in ethanol and the resulting solutions were diluted 100-fold in running medium containing 1% of FBS. After a further 10-fold dilution into the media in wells, only 0.1% of FBS was present during OCR measurements. This amount appeared to dissolve up to 25 μM of DXN at 37° C. and was well below the 1-2% limit recommended by Seahorse Bioscience. The concentration of oligomycin and the cell density used were selected based on preliminary experiments.

Measurement of Mitochrondrial Transmembrane Potential Changes Using the JC-1 Assay:

Cells were plated at a density of 80,000 cells per well in black 96-well plates and incubated for 24 hours at 37° C. (5% CO₂). They were then treated with fresh solutions of various concentrations of XN, DXN or TXN in growth medium. The final concentrations of XN, DXN, TXN were 1, 2, 5, 8, 25, 50 μM, all in quadruplicate wells, in the 96-well plates. After one hour of exposure to the compounds at 37° C. (5% CO₂), the cells were treated with 10 μL of a JC-1 staining solution (freshly prepared by diluting 11 fold 100 μL of the JC-1 agent furnished from the assay kit). After 15 to 30 minutes of incubation (37° C., 5% CO₂), the cells were washed three times with the Cell-Based Assay Buffer (prepared by dissolving three buffer tablets of the kit in 300 mL of MilliQ water). The plates were then analyzed by a fluorescent plate reader (SpectraMax Gemini XS) at 560-595 nm and 485-535 nm.

Statistical Analysis:

The cellular experiments (MTT assay and JC-1 assay) were analyzed according to ANOVA procedure, Dunnett. A p value <0.05 was considered significant.

Seahorse data were analyzed in SAS 9.2 (SAS Institute Inc., Cary, N.C.). To show a consistent baseline, OCR values (in pmol/min/mg protein) and ECAR values (in mpH/min/mg protein) for each value were divided prior to statistical analysis by the average of the four pre oligomycin treatment values (0, 8, 17, and 25 minutes) for the corresponding well and are shown as % pre oligomycin values. The original scale for OCR and for ECAR values was utilized for the OCR/ECAR ratio (in pMol/mpH). To account for repeated measures within wells over time, a repeated-measures-in-time design using ANOVA procedures in PROC MIXED was used. Fixed effects in the model were treatment (control, 2 μM DXN, 5 μM XN, 5 μM DXN, and 5 μM TXN for the compound study and control, 2, 5, 8, and 25 μM DXN for the DXN dosage study), time (0, 8, 17, 25, 34, 43, 52, 60, 69, 78, 86, and 95 minutes), and their interaction. The random effect was the experiment (5 experiments for the compound study and 3 experiments for the DXN dosage study). To account for repeated measures within wells over time, a first order homogeneous variance-covariance matrix [AR(1)] was fitted for each well. Using the ESTIMATE statement, a priori contrasts were constructed by comparing the changes in compound treated cells from the last two time points prior to compound treatment (average 52 and 60 minutes) to the changes in control cells during the same time period. This was done for each compound and dosage for 69, 78, 86, and 95 minutes separately and for all four time points combined. Results are shown in the text and the graphs as least squares means and their corresponding SEM (standard error of mean). A P-value of 0.05 was considered statistically significant and is shown as star in the graphs (FIG. 5).

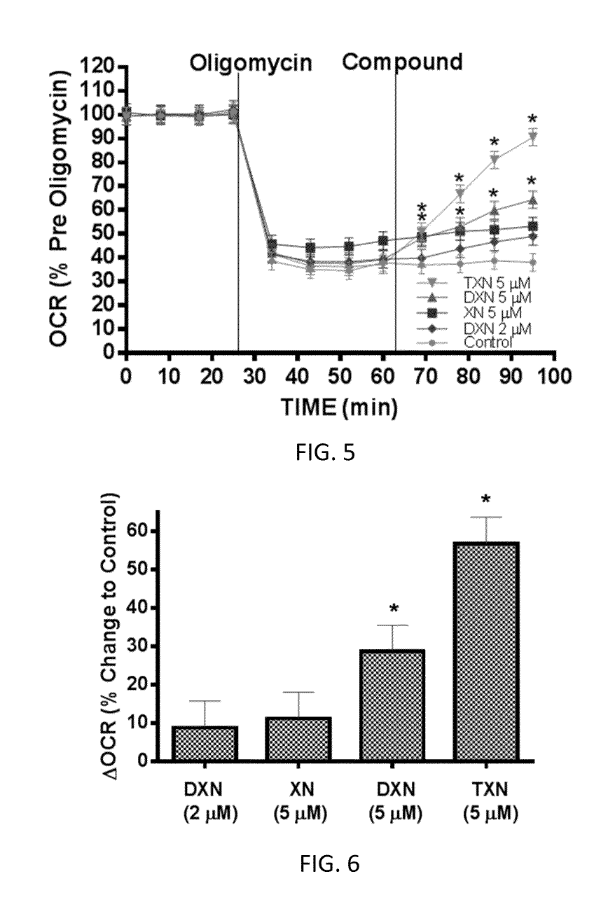

The bar graphs showing the xanthohumol-induced change in OCR, ECAR, and OCR/ECAR, graphed as % change to control (FIG. 6), are calculated as follows: first, using the trapezoidal rule, the area under the curve pre oligomycin treatment (0 to 25 min), post oligomycin treatment (25 to 60 min, and post compound treatment (60 to 95 min) was calculated. Next the ATP-associated OCR fraction {[(post oligomycin OCR/pre oligomycin OCR)−1]×100}, the glycolysis reserve fraction {[(post oligomycin ECAR/pre oligomycin ECAR)−1]×100}, and the compound-induced changes in OCR, ECAR, and OCR/ECAR {[(post xanthohumol OCR/ECAR, or OCR/ECARCompound/post oligomycin OCR/ECAR, or OCR/ECARCompound)−1]×100} was calculated. The calculated values were used for the statistical analysis in PROC MIXED. The fixed effect in the model was treatment (as described previously) and the random effect was experiment (as described previously). Using the ESTIMATE statement, a priori contrasts were constructed by comparing the compound induced changes to the changes in control cells during the same time period. Those least squared means estimates and SEM, displayed as % change to control, are shown in the text and graphs. For the calculation of the area 21, only the two last values between 52 and 60 min were considered. (Control, 2 μM DXN, 5 μM XN, 5 μM DXN, and 5 μM TXN for the compound study and control (FIG. 6), 2, 5, 8, and 25 μM DXN for the DXN dosage study fitted to the same statistical model as described in the previous paragraph with the exception that time and its interaction was not included in the model (FIG. 13). A P-value of 0.05 was considered statistically significant and is shown as star in the graphs.

MTT Assay:

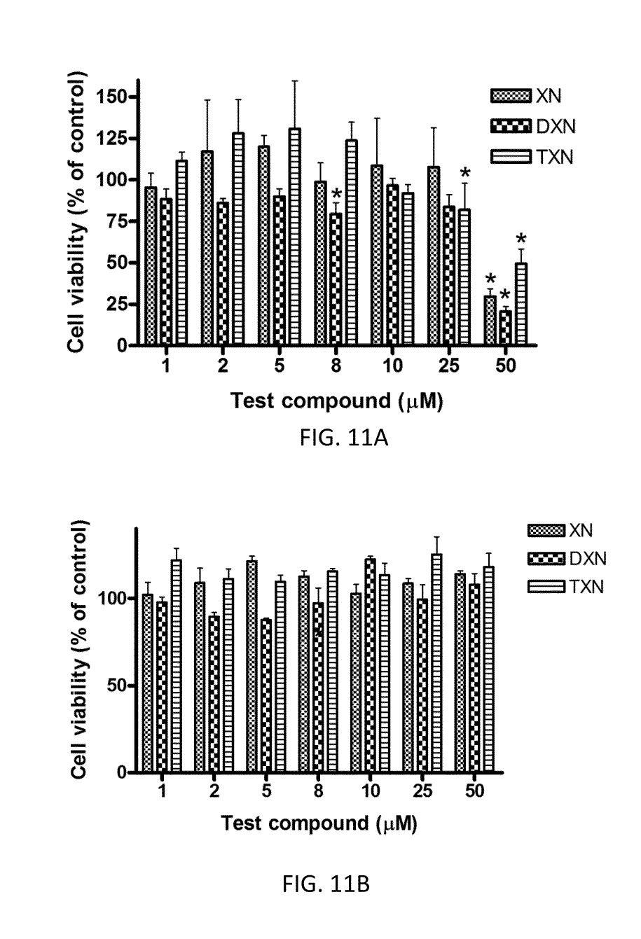

Two MTT assays were conducted (FIGS. 11A and 11B): after one hour and 24 hours of exposure. After one hour of exposure, XN, DXN and TXN did not significantly affect cell viability up to 50 μM. After 24 hours of exposure, cell viability was decreased at 8 μM of DXN, 25 μM of TXN and 50 μM of XN.

Optimization of Seahorse Assay Conditions (Cell Density, Oligomycin and FCCP Concentrations):

As advised by Seahorce Bioscience, running a cell titration assay with FCCP at different concentrations prior to any Seahorse experiment is an important step to determine proper cell density for the cell line used. Then, oligomycin was tested at different concentrations at the proper cell density in order to determine the optimal working concentration. These optimization and titration assays offered starting conditions in determining the uncoupling effects on the mitochondria.

In the cell titration assay, cells were plated at 10,000, 20,000 and 40,000 cells/wells and FCCP was injected at 0.3 μM or 0.5 μM. The maximal OCR was obtained at a density of 40,000 cells/well at a concentration of 0.5 μM of FCCP. The values of OCR obtained were between 205 and 234 picomole/minute for the control baseline. An increase was observed after injection of FCCP: 517 to 498 picomole/minute and 605 to 518 picomole/minute for 0.3 μM of FCCP and for 0.5 μM of FCCP, respectively. The values of OCR obtained at this density were in the range of the typical values obtained for C2C12 muscle mouse cells so the density of 40,000 cells/wells was chosen for the following experiments. After injection of FCCP, especially at 0.5 μM, the cells increased their OCR, meaning that they behaved as expected in the presence of a mitochondrial uncoupler. DXN was also injected at 2 μM at 20,000 cells/well to have an idea of its potential effects on OCR. An increase of OCR of 204% of the baseline was observed.

In the oligomycin optimization assay, oligomycin was injected at 0 μM, 0.5 μM 1 μM and 2 μM followed by an injection of 0.5 μM of FCCP. After injection of oligomycin, the resulting decrease in OCR was the most significant at a concentration of 1 μM. This concentration was thus the optimized working concentration for the next experiments.

Methods of Making the Compounds:

In some examples, Wilkinson catalyst (RhCl(PPh₃)₃) was used to selectively achieve 1,4-conjugate reduction of the double bond product over the 1,2-reduction of the carbonyl product (see mechanism of the catalyzed hydrogenation in FIG. 19). Without being limited to a single theory of operation, it is currently believed that the double bond of the α,β-unsaturated carbonyl compound can be more reactive than the prenyl group because the phenol proximity makes the reactivity site planar and thus the Wilkinson catalyst can better attack. The ionic liquid, [bmim][BF4], was used as solvent and ammonium formate as the source of hydrogen. Ionic liquids have the advantages to have a good solvent power for many organic chemical reactions, a high miscibility, a high thermal stability, low vapor pressure and other interesting physical properties.

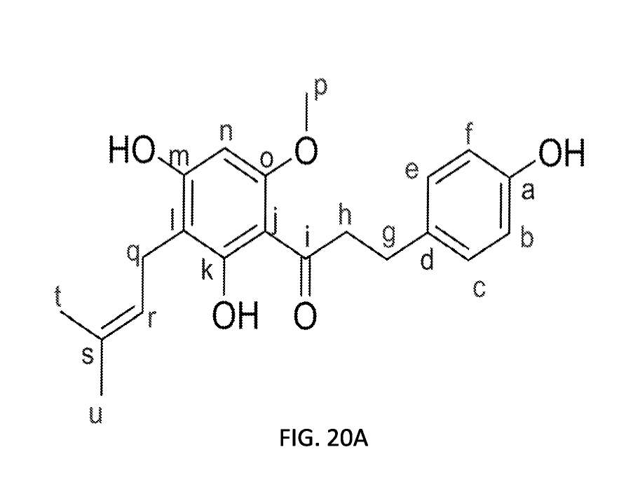

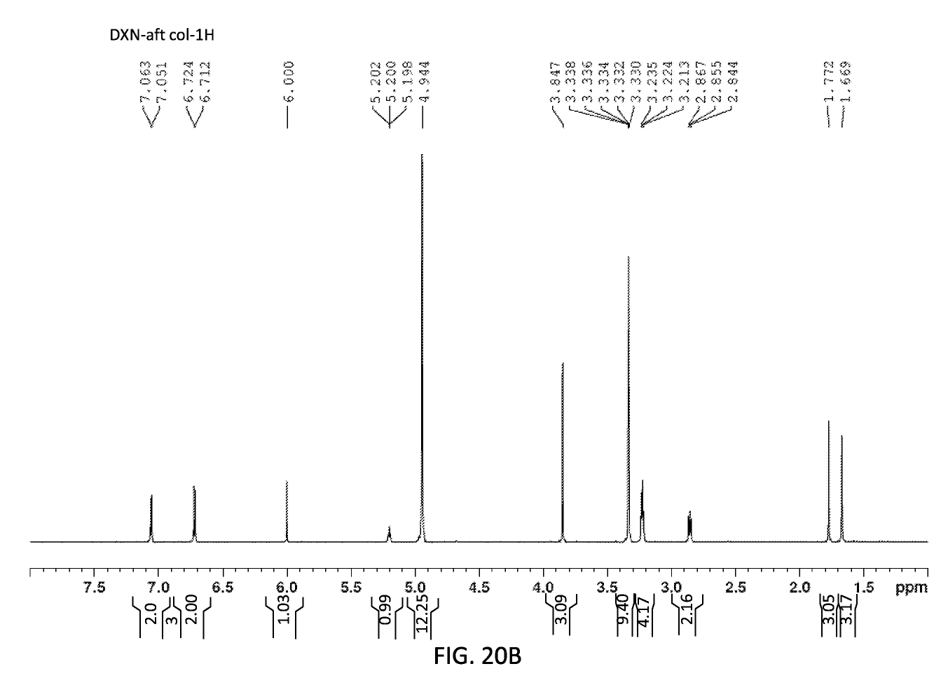

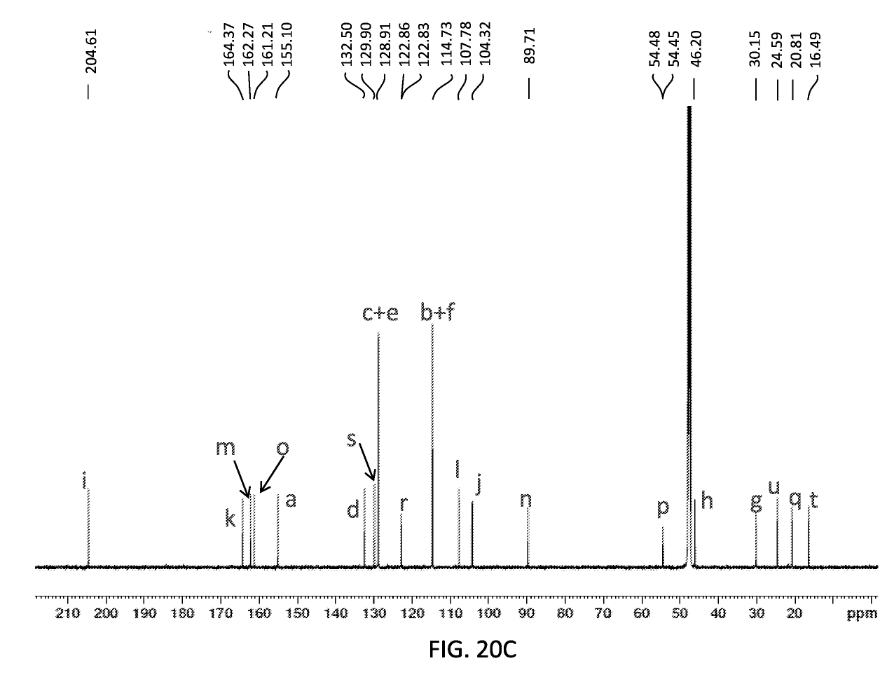

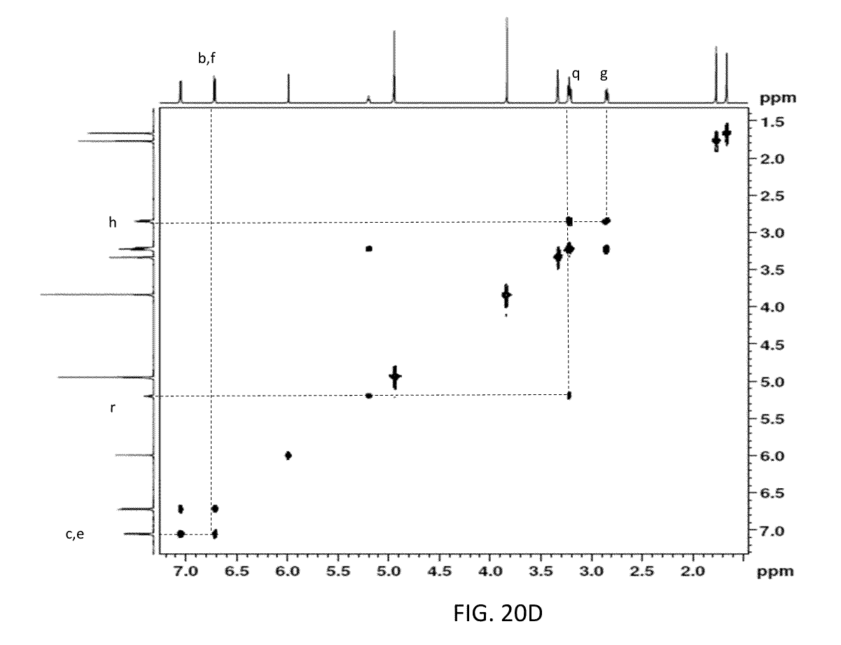

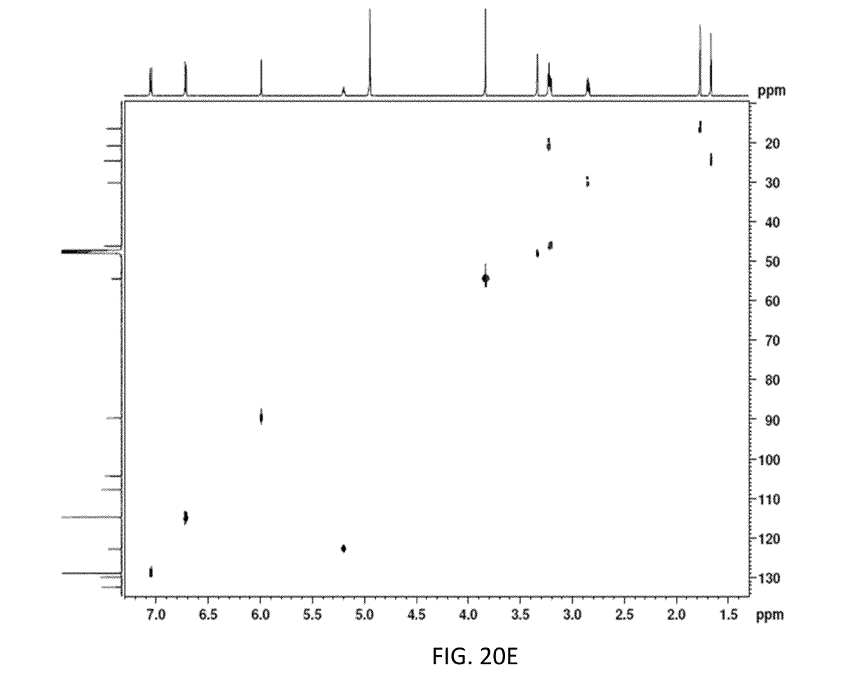

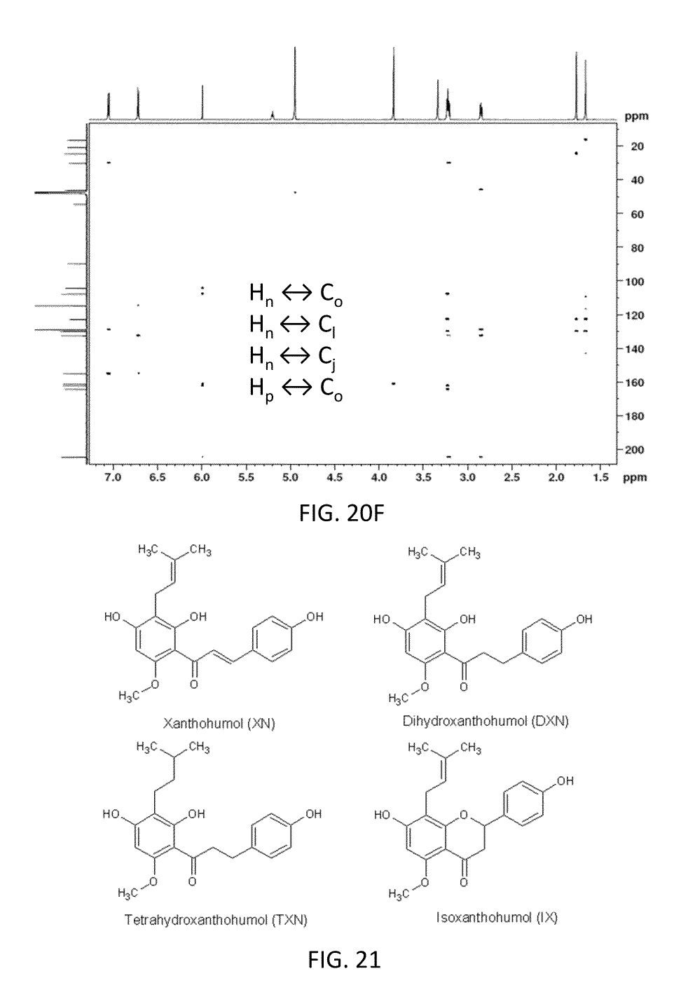

DXN was made using catalytic hydrogenation with the Wilkinson catalyst method by stifling the reaction mixture at 90° C. for 3 hours. Various combinations of organic solvent or organic solvent with ionic liquid were evaluated. In some embodiments, a reaction system of 10% mol of RhCl(PPh₃)₃, ammonium formate (4 eq), and [bmim][BF4] ionic liquid (6.3 eq, 8.5 mmol) was used, which showed a 89% conversion of XN into DXN by HPLC. The reaction mixture was extracted with ethyl acetate and washed with water and the organic layer was dried with anhydrous sodium sulfate (Na₂SO₄) and evaporated under reduced pressure. The crude product was purified by flash chromatography on a silica gel column eluted with ethyl acetate: hexane (1:1.7, v/v) as eluent. DXN was obtained as a pale yellow solid in an isolated yield of 44%. The compound was characterized by NMR and MS-MS analysis (FIGS. 20A-20F). 4 g of DXN was successfully synthesized and purified to be used for animal studies. In yet other examples, the isolated yield of DXN was increased to 62% by extracting the reaction mixture with diethyl ether.

In some examples using a palladium-catalyzed hydrogenation method, the formation of an undesired cyclic product by intramolecular rearrangement of XN under acidic condition. In yet other examples, the use of toluene and dichloromethane resulted in low yields due to the poor solubility of XN in these solvents.

### Example 1

Comparison of the Uncoupling Effects of XN, DXN, TXN:

80% of the oxygen consumed by the cells is due to mitochondrial respiration. The ATP synthase is the major pathway whereby protons reenter the mitochondrial matrix and it is also coupled to the respiration chain. Therefore, cellular OCR was measured after injection of the ATP synthase inhibitor, oligomycin, to determine whether the addition of either XN, DXN or TXN could stimulate the OCR, when ATP synthesis is inhibited. A diluted solution of ethanol in the running medium (0.01% final concentration in wells) was used as a vehicle control in order to measure the significance of the impacts of the compounds on the OCR. At 5 μM concentration, it was observed that DXN and TXN were able to significantly increase the OCR in the presence of oligomycin: by 28.7±6.9% for DXN and by 56.8±6.9% for TXN, respectively, compared to the baseline and for an average of five independent experiments (see FIGS. 5 and 6). A concentration of 5 μM appeared to be sufficient for both DXN and TXN to have a significant effect on the OCR. TXN was found to be most potent as a mitochondrial uncoupler among our three compounds. XN did not demonstrate a significant increase in the OCR at 5 μM in contrast to a previous study investigating its mitochondrial uncoupling effects. The effects of DXN at 2 μM were similar to the effects of XN at 5 μM (on average, 11.3±6.9% and 8.9±7.0% for XN at 5 μM and DXN at 2 μM, respectively, FIG. 6).

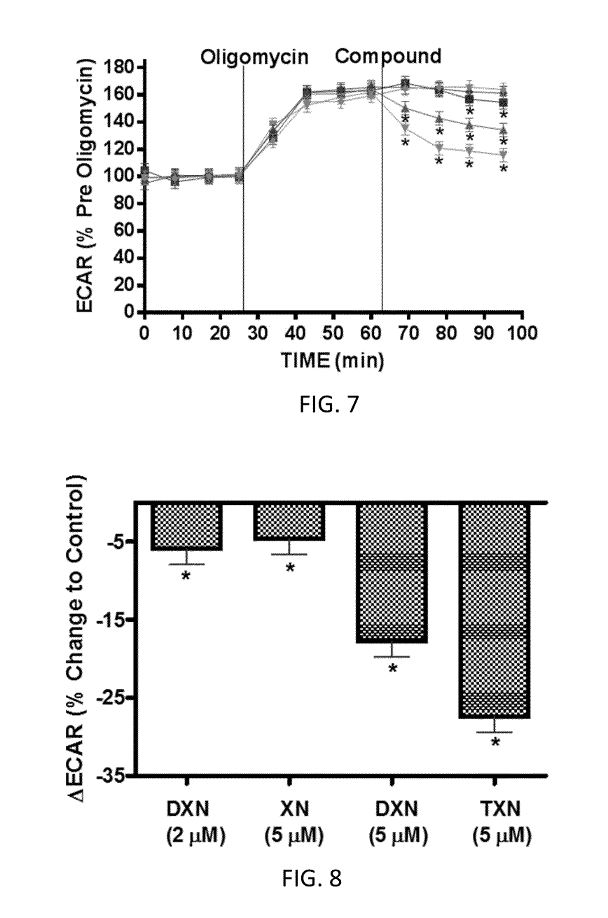

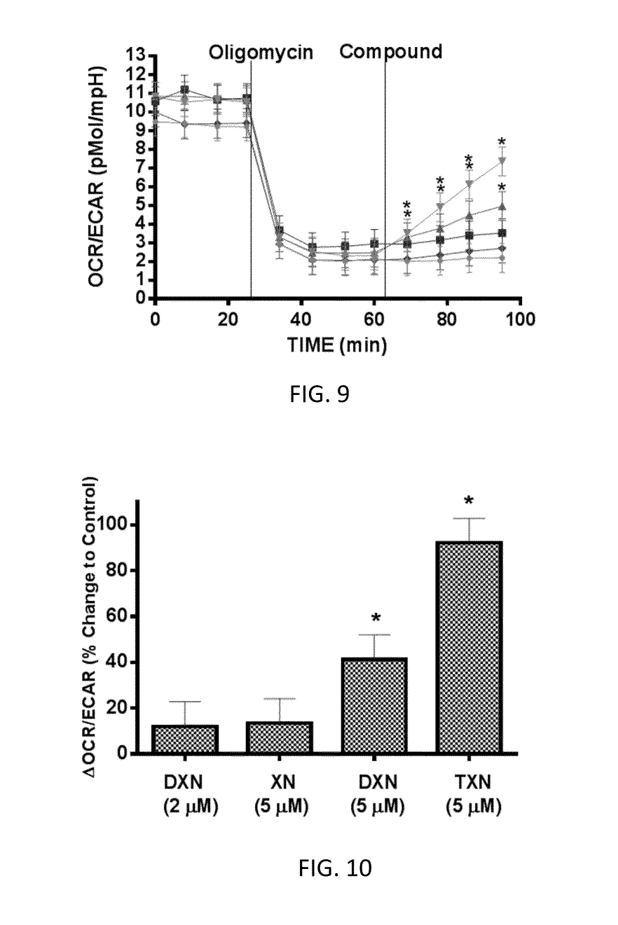

After injection of oligomycin in each well, the ECAR increased until reaching a plateau with the value 160.1±5.1% (FIGS. 7 and 8). The injection of 5 μM of XN, DXN and TXN and 2 μM of DXN resulted in a significant decrease in ECAR of 4.7±2.0%, 17.8±2.0%, 27.5±2.0%, 5.9±2.0% respectively, for an average of five independent experiments. Additional exemplary results are illustrated in FIGS. 9 and 10.

Dose-Response Effect of DXN:

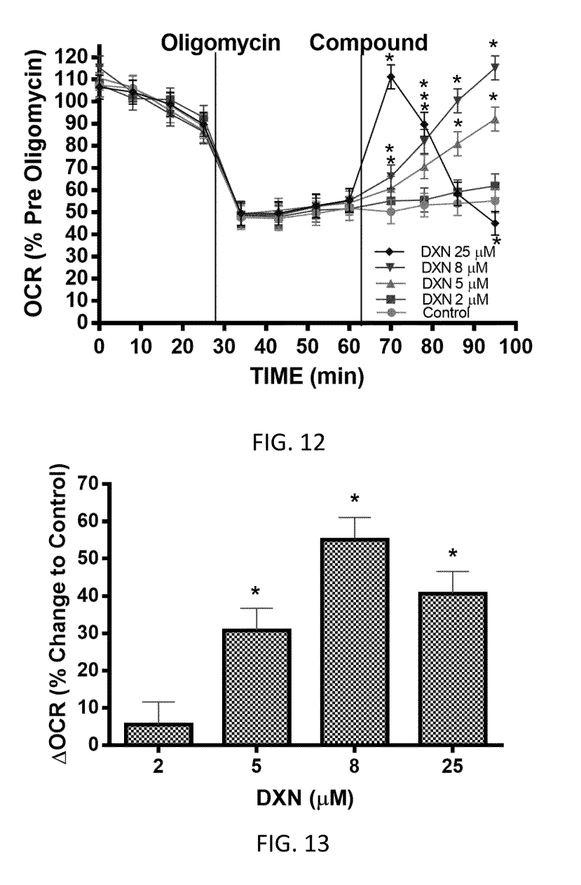

Different concentrations (2, 5, 8, 25 μM) of DXN were tested on the same plate. A dose-effect relationship (FIGS. 12 and 13) was observed: the greater the dose, the greater the OCR was increased in the presence of oligomycin, i.e., by 6±6%, 30.8±6.0%, 55.1±6.0% at a concentration of 2 μM, 5 μM and 8 μM, respectively, for an average of three independent experiments. The concentrations of 5 μM and 8 μM thus showed a significant OCR increase. At higher dose (25 μM), the cells exhibited an initial increase in the OCR that was immediately followed by a sharp decrease.

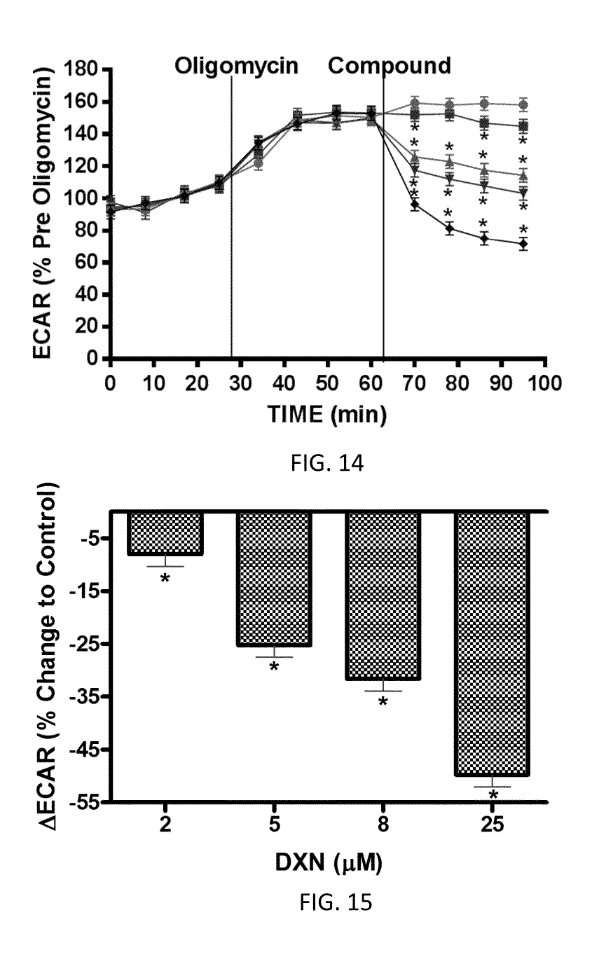

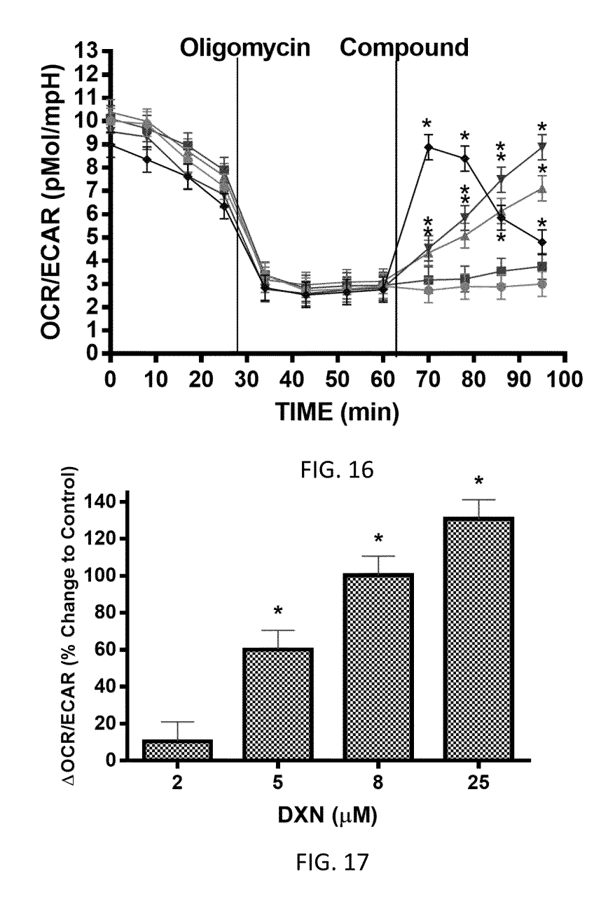

After injection of oligomycin into each well, the ECAR increased until reaching a plateau with the value 151.1±3.4% (FIG. 14). The injection of 2 μM, 5 μM, 8 μM and 25 μM of DXN resulted in a significant decrease in ECAR of 8.0±2.4%, 25.2±2.3%, 31.6±2.3, 49.8±2.3%, respectively (FIGS. 14 and 15). Additional exemplary results are illustrated in FIGS. 16 and 17.

JC1 Assay:

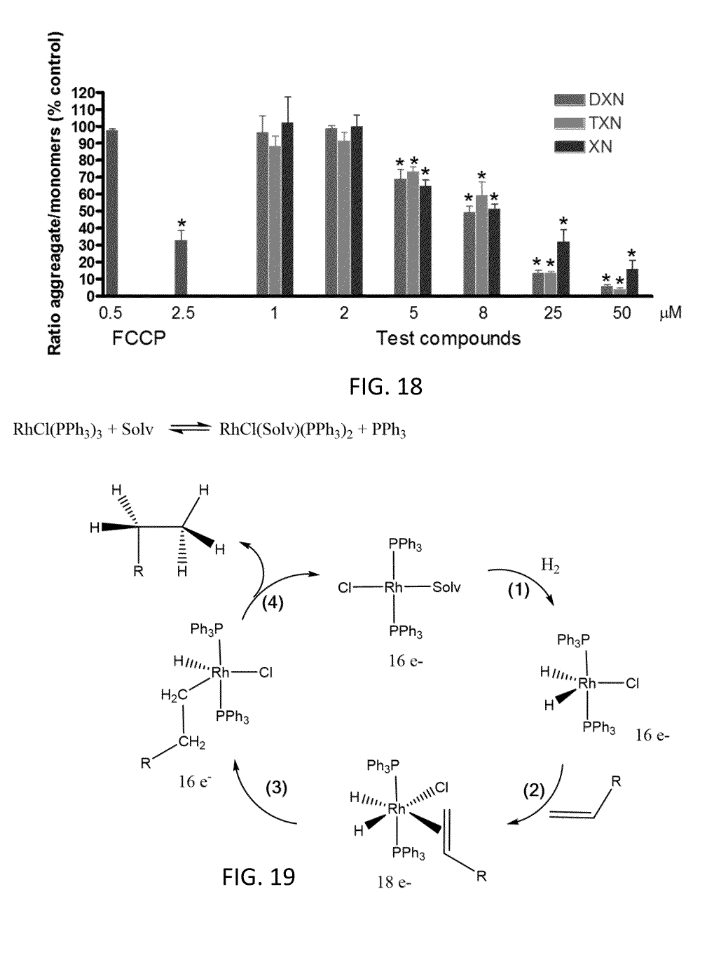

To determine whether XN, DXN and TXN were acting as protonophores, i.e., being able to transport protons across the mitochondrial inner membrane instead of transport via the proton channel of the ATP synthase, the mitochondrial membrane potential variations were measured. Mitochondria are especially responsible of key events in health and apoptosis cells. While the proton motive force (Δp) drives ATP synthesis, the electrical component, Δψm, is primarily responsible for the generation of ROS and NADH reduction. Alteration of the mitochondrial activity results in a depolarization or loss of mitochondrial transmembrane potential. Δψm is thus an important parameter of mitochondrial function and can be used as an indicator of cell health. Qualitative variations, like a loss of Δψm, can be studied by evaluating the changes in fluorescence intensity of cells stained with cationic dyes such as rhodamine-123 or the new cytofluorometric lipophilic cationic dye, 5,6-dichloro-2-[3-(5,6-dichloro-1,3-diethyl-1,3-dihydro-2H-benzimidazol-2-ylidene)-1-propen-1-yl]-1,3-diethyl-1H-benzimidazolium, monoiodide (JC-1). JC-1 can selectively enter into mitochondria and reversibly change color from green to red as the membrane potential increases. In healthy cells with high mitochondrial transmembrane potential, JC-1 spontaneously forms complexes known as J-aggregates with intense red fluorescence with excitation and emission at 560 nm and 595 nm, respectively. In apoptotic or unhealthy cells with low potential difference (or in our case of a dissipation of the proton gradient), JC-1 remains in the monomeric form, which shows only intense green fluorescence with excitation and emission at 485 nm and 535 nm, respectively. The ratio of fluorescence intensity of J-aggregates to fluorescence intensity of J-monomers can be used as an indicator of cell heath or mitochondrial state. After treatment with various concentrations of the compounds and spectrometric measurements, the ratios of aggregates to monomers (expressed as percent of control) were obtained (FIG. 18). A decrease in the values of the ratios compared to the control is indicative of an alteration in the potential across the mitochondrial membrane. The ratios changed significantly from the control values for XN, DXN, and TXN at 5 μM, 8 μM, 25 μM and 50 μM. The higher the dose, the greater the loss of transmembrane potential. To confirm the directional dye behavior, a group with different concentrations (0.5 μM and 2.5 μM) of the protonophore FCCP was treated, which was able to collapse the charge gradient at 2.5 μM with a ratio value of aggregates to monomers of 33.0±5.6%.

XN, DXN and TXN were demonstrated to act as mitochondrial uncouplers on C2C12 cells by their ability to increase significantly the OCR after selective inhibition of the ATP synthase by oligomycin. In some embodiments, determining DXN dose response and comparison of XN, DXN and TXN provided results consistent with each other: DXN effect on the OCR increased with the dose injected and TXN was more potent than DXN, but DXN was more potent than XN.

80% of the oxygen consumed by the cells is due to mitochondrial. After inhibition of the respiratory chain by oligomycin, the 51.4±6.3% residual OCR (value for the DXN dosage experiment) corresponded to the non-coupled to ATP synthesis (proton leak and cytosolic respiration, see FIG. 4). 49% of the OCR was thus coupled to oxidative phosphorylation. These values indicate that 54% of the total OCR is coupled to ATP synthesis.

The doses tested were low enough to get a mild mitochondrial effect in order to reduce the proton motive force and to expend energy without showing toxicity. A cell viability experiment (MTT assay) was conducted prior to the Seahorse experiments to verify that the concentrations used (5 μM for all compounds and 2 to 25 μM for DXN dose-response) were not toxic to the cells after one hour of test compound exposure. However, at higher dose (25 μM), the cells exhibited an initial increase in the OCR that was rapidly followed by a sharp decrease. Without being limited to a particular theory of operation, it is currently believed that this finding can be explained as a response to a toxic insult. In addition, higher concentrations of XN and TXN tended to inhibit OCR. In fact, at higher concentrations, the cells rapidly increased their OCR but they were not able to maintain the OCR due to toxicity because the mitochondrial integrity is essential for the cell viability. However, 25 μM was not toxic to cells in the MTT assay. MTT assay is based on the ability of the cells to reduce the MTT complex exclusively via mitochondrial succinic dehydrogenases. However, MTT conversion has been shown to be a complex process dependent on multiple factors (critical role of cell membrane function for example). If DXN, TXN and XN act on the cell membrane, they will thus have effects on MTT uptake and excretion. The MTT assay has been also shown to be less sensitive to alteration of ETC activity than some respirometry analysis.

XN, DXN and TXN have shown to act as mitochondrial uncouplers able to depolarize the mitochondrial transmembrane membrane. The mechanism of action of the uncouplers can be a direct effect (by acting as protonophores dissipating the proton gradient) or an indirect effect by stimulating UCP expression. The influence of XN and its derivatives on UCP activity could be investigated by using UCP-knockout and UCP-overexpressed mices. The induction of UCP-expression would require time (interactions with translation proteins processes). However, in the Seahorse assays, the uncoupling effect is immediately observed after injection of the uncoupling compounds so the effect of XN derivatives must be direct and not UCP-dependent.

In the Seahorse experiments conducted, the injection of oligomycin induced a decrease in the OCR and an increase in the ECAR. The cells were not able anymore to supply ATP via oxidative phosphorylation so they shifted to glycolysis, an anaerobic process, to provide energy. Glycolysis generates pyruvate that can be converted to lactate. In animals, lactate is converted back to pyruvate that can normally enter the citric acid cycle to be oxidized and generate energy and electrons flowing through the respiration chain. In the case of an excess of lactate, when it cannot be converted and enter the citric acid cycle that does not work, the environment is acidified (the pH decreases) because of the presence of lactic acid, and the ECAR increases. After injection of the uncoupler DXN, the respiration chain is uncoupled and is working versus the oxidative phosphorylation, the OCR increases and some heat is produced by dissipating the proton gradient (see FIG. 8). Pyruvate is able to enter the citric acid cycle so less of this substrate is converted to lactate resulting in a light decrease in the ECAR compared to prior injection of DXN. ATP is still produced though, since ECAR is not equal to zero, allowing the cells to live. The cells are continuously trying to restore the energetic equilibrium. In reality and in therapies, the uncoupler is added without prior injection of ATP synthase inhibitor. There is thus still some production of ATP through oxidative phosphorylation (but less than usual) and there is a continuous competition between the flow of protons through ATP synthase forming ATP and the dissipation of the proton motive force via the uncoupler generating heat. An increase in ECAR is expected since the cells will shift to glycolysis to supply the cells in the amount of energy needed, with injection of 2 μM of DXN (FIG. 4).

In some embodiments, the mitochondrial uncoupling effect of xanthohumol can be increased by synthetic conversion into α,β-dihydroxanthohumol and tetrahydroxanthohumol. It was confirmed that xanthohumol and its two derivatives are able to dissipate the proton gradient, possibly by acting as protonophores, thereby increasing cellular oxygen consumption without ATP production. The significance of these findings is that xanthohumol and its synthetic derivatives increase energy expenditure and may be used therapeutically to treat metabolic syndrome, or risk factors associated with metabolic syndrome, such as obesity, diabetes, and/or cardiovascular disease. In some embodiments, DXN has the advantage over XN in that it lacks pro-estrogenic effects and reactivity towards proteins. In other embodiments, DXN has the advantage over TXN in that its mitochondrial uncoupling effects are milder and may therefore be less toxic.

### Example 2

The following materials and methods were used to carry out the in-vivo experiments described in this example.

Cell Culture:

Cellular uptake investigations were performed using C2C12 mouse myocytes. Cells were cultured in 75 cm2 flasks using Dulbecco's Modified Eagle Medium (DMEM) obtained from Sigma-Aldrich, which was supplemented with 10% Fetal Bovine serum (FBS) and 1% penicillin/streptomycin solution (PS). Cultures were maintained in an incubator at 37° C. and 5% CO2.

Cellular Uptake of XN, DXN and TXN:

Uptake comparisons were conducted by seeding three 12-well plates with 1 mL of C2C12 cells in suspension with a density of 100,000 cells/mL, which were then incubated until confluent. Four treatment groups were compared, including XN, DXN, TXN and a no compound control. Each compound was tested in quadruplicate in the presence of cells and in medium not exposed to cells. Medium not exposed to cells was examined for compound concentration to help account for protein binding in medium. Confluent cells were subsequently treated with fresh DMEM containing 1% FBS, 1% PS, as well as 5 μM XN, DXN, or TXN and incubated at 37° C. and 5% CO2 for one hour. Following incubation, cell medium was collected and the cells were washed once using Hanks Balanced Salt Solution (HBSS). HBSS was then aspirated, and 0.5 mL of cell extraction buffer (50% methanol:ethanol, v:v) was added to each well and the plates were covered with a plastic adhesive plate cover. The plates were placed in a freezer and maintained at −80° C. for 24 hours. After freezing, the plates were removed from the freezer and the cells were removed from the wells using a cell scraper and collected. An additional 0.5 mL of extraction buffer was added to each well as a rinse, which was collected and added to the cell extracts. Samples were centrifuged for 5 minutes at 13,000 g, and the supernatant was placed in liquid chromatography tandem mass spectrometry (LC-MS/MS) vials for analysis. Cell medium samples were prepared by 1:4 dilution with acetonitrile (ACN) and subsequent centrifugation for 5 minutes at 13,000 g. Supernatants were transferred to LC-MS/MS vials. A volume of 20 μL of 2 μM 2,4′-dihydroxychalcone (DHC) in ethanol (EtOH) was added to all LC-MS/MS samples as an internal standard.

Mass Spectrometry:

All cell culture samples were analyzed by LC-MS/MS using an Applied Biosystems API 4000 Q-trap triple quadrupole mass spectrometer. Analytes were separated by high performance liquid chromatography (HPLC) using a Phenomenex pentafluorophenyl (PFP) Luna 5 μm column. Once concentrations could be determined, mass balance of XN, DXN or TXN was calculated based on the average total volume of the sample, approximately 0.995 mL for cell medium, and 0.75 mL for cellular extracts.

Mouse Feeding and Maintenance:

The total number of C57BL6/J mice was 48, with 12 mice belonging to each of 4 treatment groups. Duration of the feeding was 14 weeks. Mouse feed contained high fat content (60% of kCal), and was supplemented with XN, DXN, or TXN in the treatment groups. XN, DXN and TXN content within the mouse diet were designed to deliver a dose of 30 mg/kg/day body weight. Food intake was measured three times per week by weighing the food pellet before placement in the cage, and at the time of feed changing by weighing uneaten food. Mice were unrestricted in dietary intake, being allowed to eat as much food as desired. Mice were weighed once per week. Groups included low dose (30 mg/kg) XN, DXN, TXN and no supplement control. Mice were maintained in accordance with the Oregon State University Institutional Animal Care and Use Committee (IACUC) regulations. One mouse was maintained on a standard AIN-93M chow diet to be used as a healthy reference during glucose tolerance assays.

Mouse Glucose Tolerance Testing:

Glucose tolerance tests were conducted during week 4 and week 11 of the feeding trial. Five mice from each treatment group were used in each glucose tolerance assay. Mice were fasted for 6 hours prior to baseline blood glucose testing. Blood was collected from the mice by tail puncture, and glucose measurements were taken using a One Touch UltraMini (LifeScan, Inc.) glucometer. Mice were weighed and then given an intraperitoneal (IP) injection of glucose equal to 2 g/kg body weight. Glucose readings were taken at 0 minutes (prior to glucose injection), 15 minutes, 30 minutes, 1 hour, and 2 hours.

Plasma Metabolites:

Plasma was collected at sacrifice (14 weeks) to analyze for plasma glucose, triglycerides, cholesterol and insulin. Plasma glucose was assayed using the Wako Autokit Glucose kit (Wako Pure Chemical Industries, Ltd.). A 96-well plate (Corning Costar® assay plate, 9017) was used to combine 5 μL of plasma with 195 μL buffer solution (60 mM Phosphate buffer (pH 7.1), 5.3 mM Phenol) containing color reagent (0.13 U/mL Mutarotase, 9.0 U/mL Glucose oxidase, 0.65 U/mL Peroxidase, 0.50 mM 4-aminoantipyrine, 2.7 U/mL Ascorbate oxidase). Samples were allowed to incubate for 5 minutes, and then the absorbance of each well was measured at a wavelength of 505 nm using a SpectraMax 190 spectrophotometer. Glucose was calculated using a 6-point calibration curve containing concentrations of 0, 25, 50, 100, 200 and 400 mg/dL.

Plasma triglycerides were measured using the Infinity™ Triglycerides Reagent kit (Thermo Fisher Scientific, Inc.). 5 μL from each sample of plasma was combined with 195 μL of Infinity Triglycerides Reagent (53 mM phosphate buffer (pH 7) containing 2.5 mM ATP, 2.5 mM Mg acetate, 0.8 mM 4-aminoantipyrine, 1.0 mM 3,5-dicholoro-2-hydroxybenzene sulfonate (DHBS), 3000 U/L glycerolphosphate oxidase (GPO), 100 U/L glycerol kinase, 2000 U/L lipoprotein kinase, 300 U/L peroxidase (horseradish)) in a separate well of a 96-well plate (Corning Costar® assay plate, 9017), and allowed to incubate for 5 minutes at room temperature. Absorbance at 500 nm was then measured on a SpectraMax 190 spectrophotometer. Triglyceride levels were calculated from a 6-point calibration curve.

Plasma cholesterol was measured using the Infinity™ Cholesterol Liquid Stable Reagent assay kit (Thermo Fisher Scientific, Inc.). Five μL of plasma from each sample was combined with 195 μL of Infinity™ Cholesterol Liquid Stable Reagent (50 mM phosphate buffer (pH 6.7) containing 200 U/L cholesterol oxidase, 500 U/L cholesterol esterase, 300 U/L peroxidase (horseradish), 0.25 mM 4-aminoantipyrine, 10 mM hydroxybenzoic acid) and incubated for 5 minutes at room temperature. Cholesterol absorbance was then measured using a SpectraMax 190 spectrophotometer at 500 nm, and concentrations were calculated using a 6-point calibration curve.

Plasma insulin levels were assayed using the ALPCO 80-INSMS-E01 mouse insulin enzyme-linked immunosorbent assay (ELISA) kit (ALPCO diagnostics, Salem, N.H.). Ten μL of plasma from each sample was loaded into a 96-well plate (provided in kit) and combined with 75 μL of provided conjugate buffer. The plate was placed on a shaker and incubated for 2 hours at room temperature while shaking at 800 rpm. The buffer was then discarded and the wells were washed 5 times with 100 μL of provided wash buffer. One hundred μL of 3,3′,5,5′-tetramethylbenzidine (TMB) was then added to each well and incubated at room temperature on a plate shaker (800 rpm) for 15 minutes. After incubation, 100 μL of stop solution was added to each well, and the plate was analyzed on a SpectraMax 190 spectrophotometer at 450 nm. Sample insulin concentrations were calculated using a 6-point calibration curve.

Statistical Analysis:

Cellular uptake data was analyzed and evaluated using Microsoft Excel, and significance was determined using one-way ANOVA plus post-hoc. Data from the mouse feeding experiment was statistically evaluated using GraphPad statistical software. Differences between mouse treatment groups were determined via one-way ANOVA and Student's t-test.

Two XN derivatives, α,β-dihydroxanthohumol (DXN) and tetrahydroxanthohumol (TXN), that do not have the unsaturated ketone in their structures where evaluated (FIG. 21). Cell culture analysis of DXN and TXN revealed that they also have effects on cellular oxygen consumption, indicating that they are also likely mitochondrial uncouplers. Uncoupling effects of DXN and TXN appear to be even more dramatic than that of XN at the same concentration (5 μM), with DXN and TXN inducing 20% and 50% increases in oxygen consumption, respectively, compared to approximately 10% seen in XN. TXN's uncoupling effect is so robust in fact that the question of toxicity and dose response will need to be examined further.

In this Example, a high fat mouse diet (60% kcal) was supplemented with low doses of XN, DXN or TXN. Weight gain among mice in each group to one another and to a group of control mice receiving vehicle diet only was determined. Because XN, DXN, and TXN have such similar structures, it was unexpected that there would be such dramatic differences in metabolic effect between XN and DXN and TXN. Differences in each compound's activity or cellular uptake could provide possible explanations. For this reason, cellular uptake of each compound was compared using C2C12 mouse myocytes, and the first part of this experiment addresses this question.

Cellular Uptake:

Mass spectrometry analysis of cell contents showed uptake of XN to be approximately 6.6%, with DXN and TXN uptake at 7.8% and 6.9%, respectively. Statistical analysis did not show that differences in uptake were significant (FIG. 22).

Mouse Feeding Experiment Weight Gain:

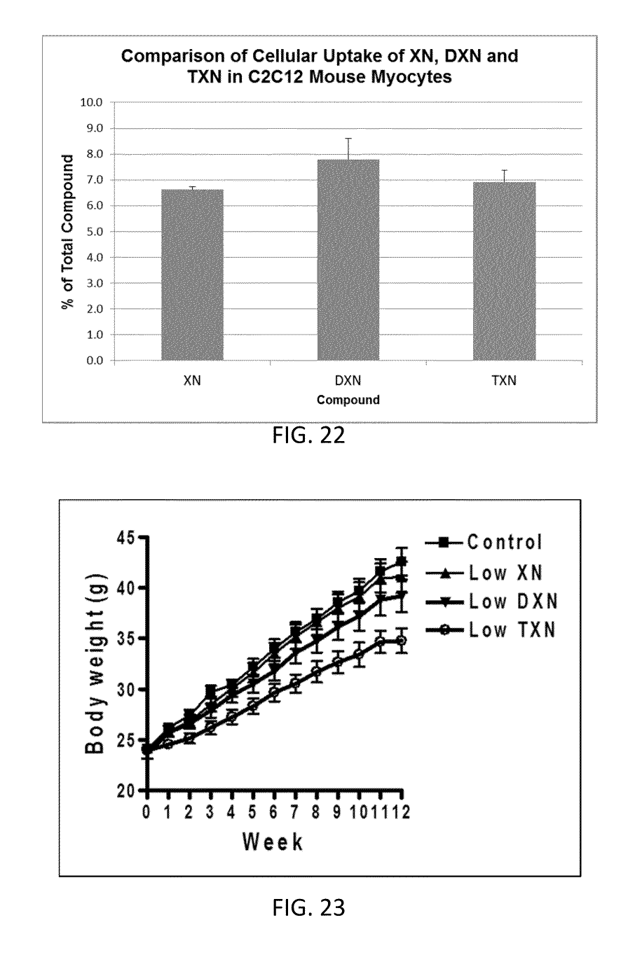

Over the course of 12 weeks, mouse weights were recorded once per week and the average weights were calculated for each group. The TXN treated mice appeared to have the most dramatic reduction in weight gain compared to the control, followed DXN, then XN (FIG. 23).

Insulin Sensitivity:

During the 4 week glucose tolerance test, average fasting blood glucose levels for all groups except TXN were partially elevated (normal range=148-210 mg/dL). Peak glucose levels were achieved by 15 minutes in the XN and TXN groups, with the average for XN at 508.8 mg/dL and 407.2 mg/dL for the TXN group. The control and DXN groups reached peak glucose levels at 30 minutes, with average blood glucose for control mice >600 mg/dL and 423.0 mg/dL for the DXN group (Table 3).

TABLE 3
Mean plasma glucose levels at time points 0, 15, 30, 60 and 120 minutes.
Glucose Tolerance Assay Mean Plasma Glucose Levels - Week 4 (mg/dL)

| Group | 0 Min | 15 min | 30 min | 60 min | 120 min |
|---|---|---|---|---|---|
| Control | 281.8 (±20.5) | 485.2 (±45.8) | 596.6 (±3.4) | 499.8 (±48.9) | 222.4 (±15.6) |
| XN | 283.4 (±4.6) | 508.8 (±28.3) | 456.0 (±41.5)* | 389.6 (±29.8)* | 167.0 (±3.8)* |
| DXN | 297.6 (±6.6) | 396.4 (±55.9) | 423.0 (±76.3)* | 342.8 (±70.4) | 176.2 (±40.0) |
| TXN | 193.6 (±17.9) | 407.2 (±38.1) | 394.6 (±50.1)* | 235.8 (±21.9)* | 149.0 (±15.3)* |

Values showing statistically significant deviation from the control are marked with * (p < 0.05).

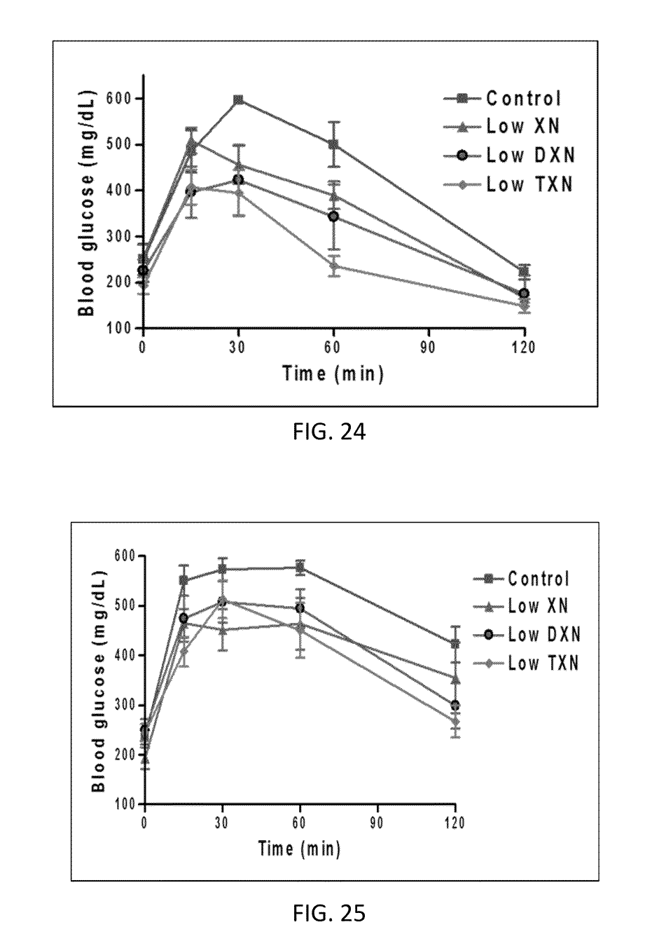

Glucose levels in all groups returned to fasting levels by 2 hours. High levels of variation occurred among DXN treated mice, though all treated mice demonstrated improved glucose recovery as indicated by less dramatic increases in plasma glucose. The fastest average recovery time was seen in TXN treated mice, though all groups had returned to their fasting glucose levels within 2 hours (FIG. 24).

Referring to FIG. 25, the week 11 glucose tolerance test showed a much more extended glucose recovery length, as well as higher glucose increases following injection. At two hours post glucose injection, average plasma glucose for all groups except TXN remained above fasting levels. Peak glucose levels occurred in XN at 15 minutes at a concentration of 465.0 mg/dL, while DXN and TXN peaked at 30 minutes, at levels of 508.0 and 513.6 mg/dL, respectively (FIG. 25). By 2 hours post-injection, average plasma glucose was highest among control mice at 422.2 mg/dL, while XN, DXN and TXN mice had averages of 373.4, 298.8, 266.8 mg/dL, respectively (Table 4).

TABLE 4
Mean plasma glucose levels measured during the week 11 glucose tolerance assay.
Glucose Tolerance Assay Mean Plasma Glucose Levels - Week 11 (mg/dL)

| Group | 0 Min | 15 min | 30 min | 60 min | 120 min |
|---|---|---|---|---|---|
| Control | 325.6 (±14.8) | 550.6 (±30.4) | 573.6 (±22.6) | 576.8 (±14.4) | 422.2 (±36.1) |
| XN | 193.2 (±21.9) | 465.0 (±30.0)* | 452.0 (±41.6)* | 463.8 (±52.2) | 373.4 (±58.1) |
| DXN | 249.8 (±22.1) | 474.4 (±46.0) | 508.0 (±41.2) | 494.8 (±38.9) | 298.8 (±45.5)* |
| TXN | 245.0 (±17.8) | 408.0 (±29.8)* | 513.6 (±37.6) | 450.8 (±5.0)* | 266.8 (±32.1)* |

Values showing statistically significant deviation from control are marked with * (p < 0.05).

Plasma Metabolic Markers:

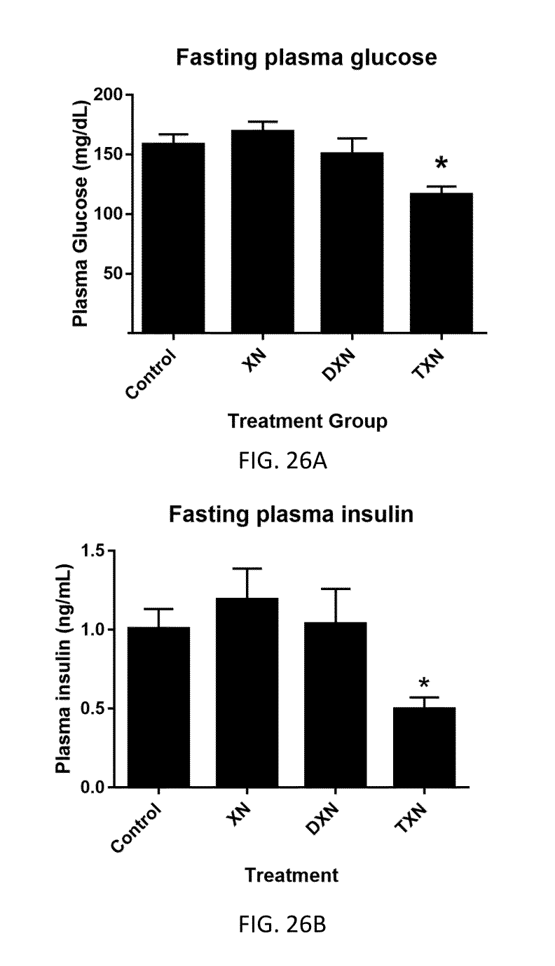

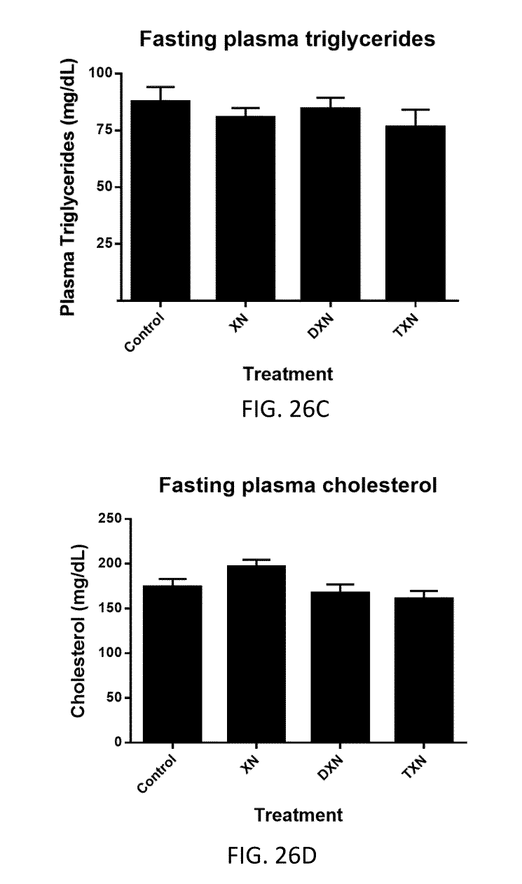

Mean fasting plasma glucose at time of sacrifice was 158.2±29.6 mg/dL for control mice, and 169.1±28.7, 150.5±45.0 and 116.1±24.1 mg/dL for XN, DXN and TXN, respectively. Average plasma triglycerides detected were 87.5±6.6, 80.7±4.2, 84.5±4.9 and 76.4±7.8 mg/dL for the control, XN, DXN and TXN groups, respectively. Plasma cholesterol averages were 173.9±31.9 mg/dL for the control, and 196.2±28.3, 167.2±33.3 and 160.7±30.7 mg/dL for XN, DXN and TXN respectively. Insulin levels averaged 1.01±0.44 ng/mL for control mice, and 1.19±0.68, 1.04±0.77 and 0.500±0.24 ng/mL for XN, DXN and TXN, respectively (FIGS. 26A-26D).

Xanthohumol (XN) and its derivatives α,β-dihydroxanthohumol (DXN) and tetrahydroxanthohumol (TXN) all show efficacy in improved insulin sensitivity as measured by the intraperitoneal glucose tolerance test, and TXN shows efficacy at reducing fasting plasma glucose and insulin levels. Corresponding reductions in weight gain also contributed to reducing the impact of metabolic syndrome. However, the dose used during this experiment was not sufficient to wholly prevent the development of diabetic-like symptoms or a pre-diabetic condition. While at 4 weeks mice receiving XN, DXN or TXN showed glucose tolerance roughly comparable to mice on a healthy diet, by 11 weeks all mice demonstrated prolonged glucose recovery times and experienced much larger increases in plasma glucose following glucose administration.

Metabolic markers such as glucose, insulin and triglycerides did not differ significantly from the controls, except in the case of TXN. A high degree of data variation hampered statistical analysis of several of these parameters, and some variation could have resulted from mice coming from different litters. Whether or not a higher dose would be more beneficial with respect to disease prevention is unclear.

The data provide evidence of a protective effect against metabolic syndrome, primarily by reduction in weight gain and enhanced insulin sensitivity. TXN appeared to have the most protective effect of the three treatments, though more information is needed regarding dose response. Thus, XN and its metabolites could prove to be effective preventative agents or assistive treatments to help those with metabolic disorder make healthy lifestyle changes.

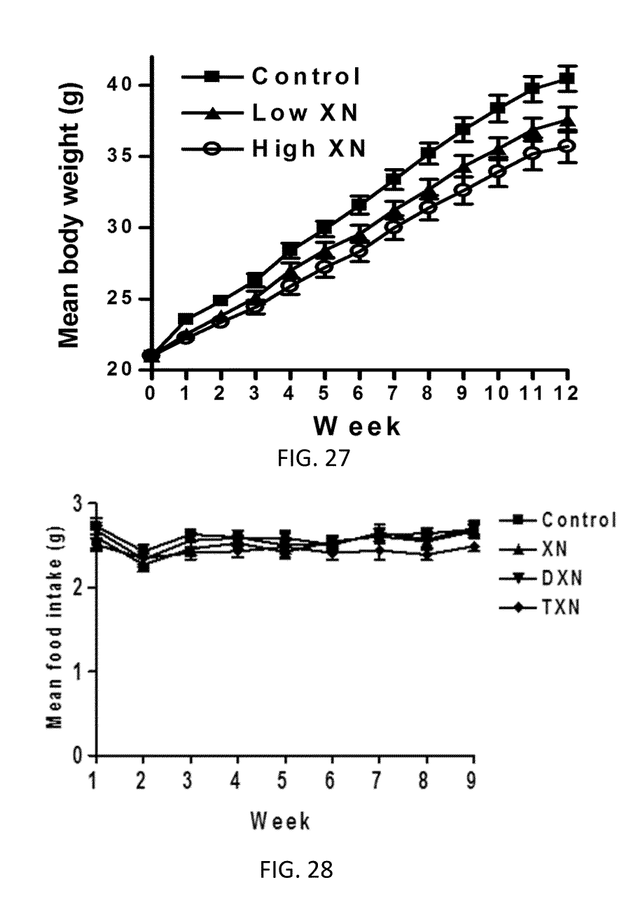

Referring now to FIG. 27, high-fat fed C57BL/6J mice gain less weight in response to oral treatment with XN at 30 (low dose) and 60 mg/kg (high dose) compared to mice treated with vehicle only (n=16/group). Data±SEM. As shown in FIG. 28, food intake was not altered.

Male C57BL/6J mice were fed a high fat diet (60% kcal fat, 20% kcal protein and 20% kcal carbohydrate) supplemented with XN, DXN or TXN at a dose of 30 mg/kg body weight/day for 14 weeks. Insulin resistance was measured via glucose tolerance assays (GTT) during weeks 4 and 11, where plasma glucose levels were measured at 15, 30, 60 and 120 minutes following an intraperitoneal glucose injection of 2 g/kg body weight. Plasma glucose, insulin, triglycerides, and cholesterol were examined following blood collection at sacrifice after 14 weeks on test diets. All mice in XN, DXN and TXN treatment groups showed improved insulin sensitivity demonstrated by smaller increases and more rapid recovery of blood glucose level compared to control (vehicle-diet) mice in the GTT. Only TXN mice showed significantly reduced fasting plasma glucose and insulin levels compared to the control, and none of the three compounds reduced plasma triglycerides or cholesterol levels. These results suggest that XN, DXN and TXN have a protective effect against metabolic syndrome by improving insulin sensitivity and glucose metabolism.

In view of the many possible embodiments to which the principles of the present disclosure may be applied, it should be recognized that the illustrated embodiments are only preferred examples of the present disclosure and should not be taken as limiting the scope of the disclosure. Rather, the scope of the disclosure is defined by the following claims. We therefore claim as our invention all that comes within the scope and spirit of these claims.

We claim:

1. A pharmaceutical composition for use in treating, ameliorating, or preventing metabolic syndrome, obesity, diabetes, and/or cardiovascular disease comprising a mitochondrial uncoupler having a Formula I 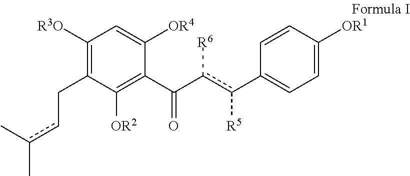
wherein each of R¹, R², R³, and R⁴ independently are selected from hydrogen, aliphatic, heteroaliphatic, haloaliphatic, heteroaryl, aryl, amide, or a prodrug; each of R⁵ and R⁶ independently are selected from halogen, thioether, ascorbic acid, or an ascorbic acid adduct; and
at least two pharmaceutically acceptable excipients selected from a fatty acid, an alkylene polyol, and a polysorbate.

2. The pharmaceutical composition of claim 1, wherein the pharmaceutical composition is formulated for administration as a dietary supplement.

3. The pharmaceutical composition of claim 1, wherein each of R¹, R², R³, and R⁴ independently is selected from hydrogen, alkyl, alkenyl, alkynyl, haloalkyl, haloalkenyl, haloalkynyl, aldehyde, ester, phosphate, phosphonate, sulfonate, or sulfonyl.

4. The pharmaceutical composition of claim 1, wherein the mitochondrial uncoupler compound is selected from any one of Formulas II-V

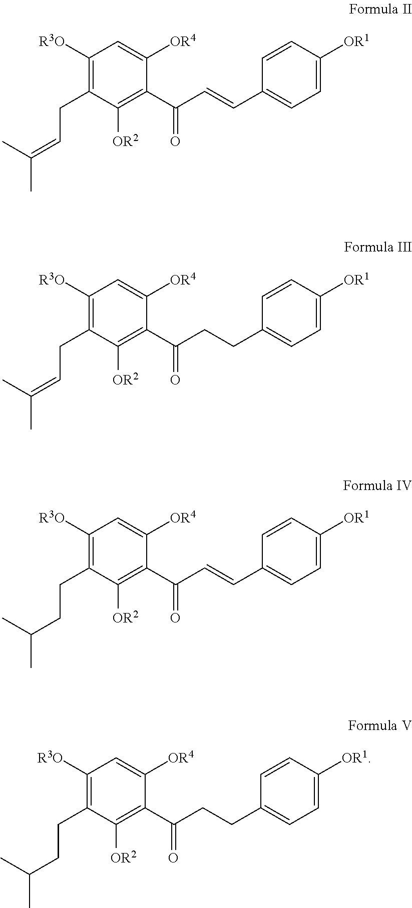

5. The pharmaceutical composition of claim 1, wherein the mitochondrial uncoupler compound is selected from 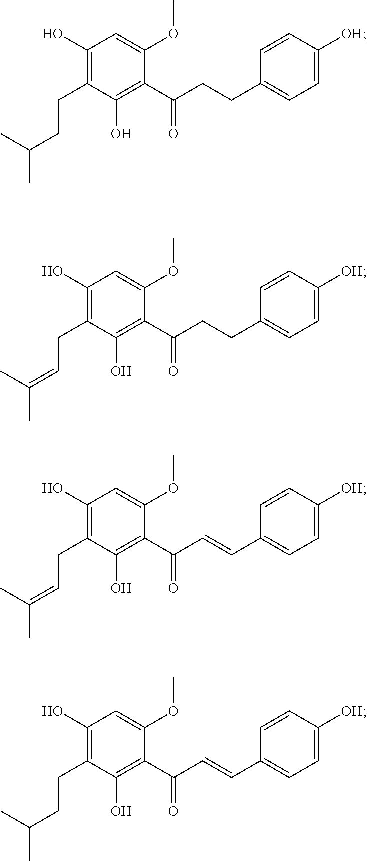 [SMILES: COc1cc(O)c(CCC(C)C)c(O)c1C(=O)CCc1ccc(O)cc1 ; COc1cc(O)c(CC=C(C)C)c(O)c1C(=O)CCc1ccc(O)cc1 ; COc1cc(O)c(CC=C(C)C)c(O)c1C(=O)/C=C/c1ccc(O)cc1 ; COc1cc(O)c(CCC(C)C)c(O)c1C(=O)/C=C/c1ccc(O)cc1]
or a hemi-succinate ester prodrug thereof.

6. The pharmaceutical composition of claim 1, wherein the at least two pharmaceutically acceptable excipients are selected from oleic acid, ethylene glycol, propylene glycol, polysorbate 80, polysorbate 60, polysorbate 40, polysorbate 20, or combinations thereof.

7. The pharmaceutical composition of claim 1, wherein the mitochondrial uncoupler compound is present in an amount ranging from 0.1 mg to 500 mg per dosage.

8. The pharmaceutical composition of claim 1, wherein the mitochondrial uncoupler compound is present in an amount ranging from 45 mg to 180 mg per dosage and the compound is

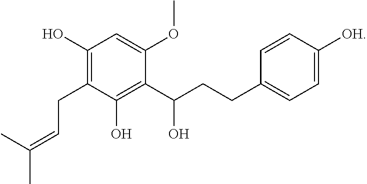 [SMILES: COc1cc(O)c(CC=C(C)C)c(O)c1C(O)CCc1ccc(O)cc1]

9. The pharmaceutical composition of claim 8, wherein the pharmaceutical composition further comprises one or more additional mitochondrial uncoupler compounds having a formula selected from one or more of

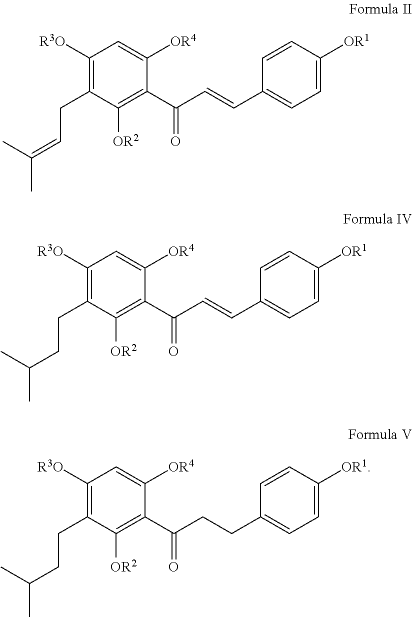

10. The pharmaceutical composition of claim 1 for treating, ameliorating, or preventing metabolic syndrome, obesity, diabetes, and/or cardiovascular disease wherein the mitochondrial uncoupler compound has a formula 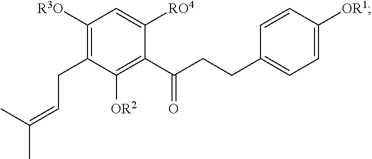
the at least two pharmaceutically acceptable excipients are selected from oleic acid, propylene glycol, and polysorbate 80; and
the composition does not comprise a drug for regulating insulin levels or a compound or extract derived from acacia.

11. The pharmaceutical composition of claim 10, wherein the mitochondrial uncoupler compound is

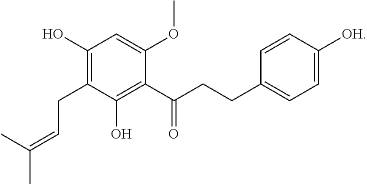 [SMILES: COc1cc(O)c(CC=C(C)C)c(O)c1C(=O)CCc1ccc(O)cc1]

12. The pharmaceutical composition of claim 1, wherein the pharmaceutical composition further comprises vitamin D.

13. A method of treating, ameliorating, or preventing metabolic syndrome, obesity, diabetes, and/or cardiovascular disease, comprising administering to a subject a pharmaceutical composition formulated to deliver a therapeutically effective amount of a mitochondrial uncoupler compound having a formula 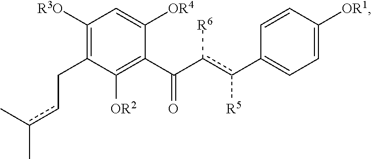
wherein each of R¹, R², R³, and R⁴ independently are selected from hydrogen, aliphatic, heteroaliphatic, haloaliphatic, heteroaryl, aryl, amide, or a prodrug; each of R⁵ and R⁶ independently are selected from halogen, thioether, ascorbic acid, or an ascorbic acid adduct; and wherein the pharmaceutical composition further comprises two or more pharmaceutically acceptable excipients selected from oleic acid, an alkylene polyol, and a polysorbate.

14. The method of claim 13, wherein the therapeutically effective amount ranges from 0.1 mg/day to 500 mg/day.

15. The method of claim 13, wherein the mitochondrial uncoupler compound has a structure

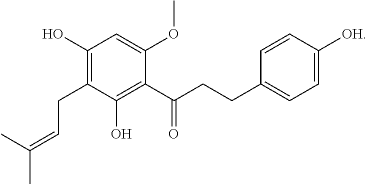 [SMILES: COc1cc(O)c(CC=C(C)C)c(O)c1C(=O)CCc1ccc(O)cc1]

16. The method of claim 13, wherein the therapeutically effective amount ranges from 45 mg to 180 mg.

17. The method of claim 13, wherein administering the mitochondrial uncoupler compound to the subject provides increased glucose tolerance.

18. The method of claim 13, wherein administering the mitochondrial uncoupler compound to the subject prevents weight gain or reduces body weight in the subject.

19. A method of making a mitochondrial uncoupler compound, comprising exposing a transition metal catalyst in an ionic liquid solution to a starting material having a formula

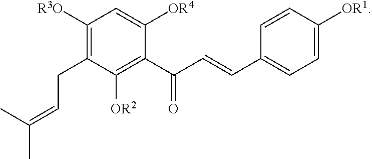

20. The method of claim 19, wherein the transition metal catalyst comprises a transition metal selected from Rh or Pd.

21. The method of claim 19, wherein the transition metal catalyst is RhCl(PPh₃)₃ and the ionic liquid comprises 1-butyl-3-methylimidazolium tetrafluoroborate.
